(19) United States
(12) Patent Application Publication
(10) Pub. No.: US 2016/0030524 A1
(43) Pub. Date: Feb. 4, 2016

(54) RECOMBINANT FACTOR VIII FORMULATIONS

(71) Applicant: BAYER HEALTHCARE LLC, Whippany, NJ (US)

(72) Inventors: De Qian WANG, Concord, CA (US); Xinghang MA, Dublin, CA (US); Nelly TSVETKOVA, Vallejo, CA (US)

(73) Assignee: BAYER HEALTHCARE LLC, Whippany, NJ (US)

(21) Appl. No.: 14/774,191

(22) PCT Filed: Mar. 11, 2014
(86) PCT No.: PCT/US14/23357

§ 371 Date: Sep. 10, 2015

Related U.S. Application Data
(60) Provisional application No. 61/799,495, filed on Mar. 15, 2013.
(60) Provisional application No. 61/869,191, filed on Aug. 23, 2013.

(51) Int. Cl. A61K 38/37; A61K 47/02; A61K 47/22; A61K 47/48

(52) U.S. Cl. CPC A61K 38/37; A61K 47/48215; A61K 47/02; A61K 47/22

(57) ABSTRACT

Provided are liquid and lyophilized recombinant Factor VIII formulations, including formulations for polymer-conjugated FVIII such as PEGylated Factor VIII.

This application claims the benefit of U.S. Provisional Application No. 61/779,495, filed Mar. 15, 2013 and U.S. Provisional Application No. 61/869,191, filed Aug. 23, 2013, both of which are hereby incorporated herein by reference in their entireties.

### BACKGROUND

Hemophilia A is caused by deficiencies in coagulation factor VIII (“FVIII”) and is the most common hereditary coagulation disorder, with an estimated incidence of 1 per 5000 males. The current treatment for hemophilia A involves intravenous injection of recombinant or plasma-derived human FVIII. Injections of FVIII are either given on demand in response to a bleeding event or as a prophylactic therapy that is administered 2 to 4 times a week. Although numerous studies have shown that prophylactic therapy decreases the complications of hemophilia A, the need for frequent intravenous injections creates barriers to patient compliance and affects patient quality of life. The requirement for frequent injections is primarily due to the short circulating FVIII half-life of 12 to 14 hours in patients.

Covalent addition of long-chain polymers, such as polyethyleneglycol (“PEG”), has been shown to increase the half-life of protein therapeutics. PEGylation is the covalent attachment of PEG molecules to proteins.

U.S. Pat. No. 5,763,401 (Nayar) discloses stable, albumin-free, lyophilized formulations of full-length recombinant FVIII (“FL-rFVIII”). U.S. Pat. No. 7,632,921 (Pan et al.) and Mei et al., Rational design of a fully active, long-acting PEGylated factor VIII for hemophilia A treatment, 116 BLOOD 270-279 (2010) disclose cysteine enhanced FVIII mutants that are covalently bound to one or more biocompatible polymers such as PEG. U.S. Pat. No. 7,087,723 (Besman et al.) pertains to albumin-free FVIII formulations. Österberg et al., Development of a freeze-dried albumin free formulation of recombinant factor VIII SQ, Pharmaceutical Research, vol. 14, No. 7 (1997), pp. 892-898 and Osterberg et al., B-domain deleted recombinant factor VIII formulation and stability, Seminars in Hematology, vol. 38, No. 2, suppl. 4 (April 2001), pp. 40-43 discuss formulations of B-domain deleted FVIII for lyophilization, including formulations containing sodium chloride, sucrose, histidine, calcium chloride dehydrate and polysorbate 80. Fatouros et al., Recombinant factor VIII SQ—influence of oxygen, metal ions, pH and ionic strength on its stability in aqueous solution, Int. J. of Pharmaceutics 155 (1997) 121-131 discloses the properties of rFVIII SQ on storage in solutions without albumin. WO2011/027152 (Jezek et al.) discloses formulations of FVIII.

### SUMMARY

In one embodiment, the invention concerns a rFVIII formulation comprising: (a) a range of from about 1 mM to about 5 mM divalent cation; (b) a range of from about 150 mM to about 250 mM sodium chloride or potassium chloride; (c) a range of from about 50 ppm to about 200 ppm of a non-ionic surfactant; and (d) a range of from about 100 IU/ml to about 5000 IU/ml of a rFVIII, wherein the rFVIII comprises an amino acid sequence that has one or more non-cysteine residues in the amino acid sequence of SEQ ID NO: 3 replaced with cysteine residues; wherein the rFVIII formulation has a pH in a range of from about pH 6.0 to about pH 7.5. In another embodiment, the invention concerns a rFVIII formulation comprising: (a) a range of from 10 mM to 100 mM MOPS; (b) a range of from 0.5% to 10% by weight of a sugar or a sugar alcohol; (c) a range of from 0.5 mM to 20 mM of a divalent cation; (d) a range of from 10 mM to 100 mM sodium chloride or potassium chloride; (e) a range of from 50 to 150 ppm of a non-ionic surfactant; and (f) a range of from about 100 IU/ml to about 1500 IU/ml of rFVIII; wherein the rFVIII formulation contains less than 5.0% by weight of components other than rFVIII having primary or secondary amine groups.

In yet another embodiment, the invention concerns a rFVIII formulation comprising: (a) a range of from 10 mM to 100 mM MOPS; (b) a range of from 150 mM to 300 mM NaCl; (c) a range of from 1 mM to 20 mM divalent cation; and (d) a range of from about 100 IU/ml to about 5000 IU/ml of nonconjugated rFVIII. In a further embodiment, the invention concerns a rFVIII formulation comprising: (a) a range of from 10 mM to 100 mM MOPS or histidine; (b) a range of from 25 mM to 200 mM NaCl; (c) a range of from 1 mM to 20 mM divalent cation; and (d) a range of from about 100 IU/ml to about 5000 IU/ml of conjugated rFVIII. In yet a further embodiment, the invention concerns a rFVIII formulation comprising: (a) about 0 mM, or a range of from about 1 mM to about 20 mM histidine; (b) a range of from 0.5% to 20% of sucrose or trehalose; (c) a range of from about 1 mM to about 5 mM divalent cation; (d) about 0 mM, or a range of from about 10 mM to about 50 mM sodium chloride; (e) about 0 mM, or a range of from about 20 ppm to about 80 ppm of a non-ionic surfactant; (f) about 0%, or a range of from about 1.0% to about 5.0%, glycine and (g) a range of from about 100 IU/ml to about 5000 IU/ml of conjugated rFVIII; wherein the rFVIII formulation has a pH in a range of from about pH 6.0 to about pH 7.5.

### BRIEF DESCRIPTION OF THE DRAWINGS

The skilled artisan will understand that the drawings, described below, are for illustration purposes only. The drawings are not intended to limit the scope of the disclosure provided herein or the scope of the claims in any way.

FIG. 1 shows schematically the domains of full-length human factor VIII and BDD-rFVIII.

FIG. 2 is a graph showing the relative turbidity of BDD-rFVIII mutants having domains linked by disulfide bonds. The turbidity was measured in buffer comprising increasing concentration of sodium chloride. Turbidity was measured by A_{340 nm}. In addition to sodium chloride, the buffer comprised 20 mM histidine, 2.5 mM calcium chloride, 29 mM sucrose, 293 mM glycine and 80 ppm polysorbate 80.

FIG. 3 is a graph showing the relative turbidity of BDD-rFVIII mutants having domains linked by disulfide bonds. The turbidity was measured in buffer comprising increasing concentration of polysorbate 80. Turbidity was measured by A_{340 nm}. In addition to polysorbate 80, the buffer comprised 20 mM histidine, 30 mM sodium chloride, 2.5 mM calcium chloride, 29 mM sucrose and 293 mM glycine.

FIG. 4 is a graph showing the relative turbidity of BDD-rFVIII mutants having domains linked by disulfide bonds. The turbidity was measured in buffer comprising increasing concentration of human serum albumin (“HSA”). Turbidity was measured by A_{340 nm}. The buffer comprised 20 mM histidine, 30 mM sodium chloride, 2.5 mM calcium chloride, 29 mM sucrose, 293 mM glycine and 80 ppm polysorbate 80.

FIG. 5 shows the relative turbidity of BDD-rFVIII mutants having domains linked by disulfide bonds. The turbidity was measured in a buffer comprising increasing concentration of sodium chloride in combination with polysorbate 80 and HSA. Turbidity was measured by A_{340 nm}. In addition to sodium chloride, HSA and polysorbate 80, the buffer comprised 20 mM histidine, 2.5 mM calcium chloride, 29 mM sucrose and 293 mM glycine.

FIG. 6 shows clarity changes for BDD-rFVIII mutants with disulfide bonds linking domains in solution before and after addition of excipients. From left to right: (1) combination of excipients (HSA, sodium chloride and polysorbate 80), (2) HSA, (3) sodium chloride, (4) polysorbate 80, and (5) before addition of HSA, polysorbate 80 and sodium chloride.

FIG. 7 is a graph showing liquid stability of full-length FVIII in histidine, MOPS and TEA buffers during 7 days storage at 40° C.

FIG. 8 is a graph showing rFVIII stability in MOPS and histidine buffer at 25° C.

FIG. 9 is a diagram showing the structure of PEGylated BDD-rFVIII. The chains protruding from the A3 region represent the PEG molecule.

FIG. 10 is a graph showing the effect of sodium chloride on the potency recovery of PEGylated BDD-rFVIII during 6 days storage at 23° C.

FIG. 11 is a graph showing the effect of sodium chloride on the potency recovery of unPEGylated BDD-rFVIII during 6 days storage at 23° C.

FIG. 12 is a graph showing normalized potency trends for PEGylated BDD-rFVIII in lyophilized Formulation A after 26 weeks. Formulation A contains 2.5 mM calcium chloride, 30 mM sodium chloride, 20 mM histidine, 293 mM glycine, 29 mM sucrose and 80 ppm polysorbate 80.

FIG. 13 is a graph showing normalized potency trends for PEGylated BDD-rFVIII in lyophilized Formulation B after 26 weeks. Formulation B contains 2.5 mM calcium chloride, 30 mM sodium chloride, 20 mM histidine, 346 mM glycine, 38 mM sucrose and 80 ppm polysorbate 80.

FIG. 14 is a graph showing normalized potency trends for PEGylated BDD-rFVIII in lyophilized Formulation C after 26 weeks. Formulation C contains 2.5 mM calcium chloride, 20 mM histidine, 234 mM sucrose and 80 ppm polysorbate 80.

FIG. 15 is a graph showing normalized potency trends for PEGylated BDD-rFVIII in lyophilized Formulation D after 26 weeks. Formulation D contains 2.5 mM calcium chloride, 20 mM histidine, 211 mM trehalose and 80 ppm polysorbate 80.

FIG. 16 is a graph showing normalized potency trends for PEGylated BDD-rFVIII in lyophilized Formulation A up to 30 months. Formulation A contains 2.5 mM calcium chloride, 30 mM sodium chloride, 20 mM histidine, 293 mM glycine, 29 mM sucrose and 80 ppm polysorbate 80.

FIG. 17 is a graph showing normalized potency trends for PEGylated BDD-rFVIII in lyophilized Formulation B up to 13 weeks. Formulation B contains 2.5 mM calcium chloride, 30 mM sodium chloride, 20 mM histidine, 346 mM glycine, 38 mM sucrose and 80 ppm polysorbate 80.

FIG. 18 is a graph showing the normalized potency trends for PEGylated BDD-rFVIII (200 IU/mL) in lyophilized Formulation A up to 12 months. Formulation A contains 2.5 mM calcium chloride, 30 mM sodium chloride, 20 mM histidine, 293 mM glycine, 29 mM sucrose and 80 ppm polysorbate 80.

FIG. 19 is a graph showing the normalized potency trends for PEGylated BDD-rFVIII (1200 IU/mL) in lyophilized Formulation A up to 9 months. Formulation A contains 2.5 mM calcium chloride, 30 mM sodium chloride, 20 mM histidine, 293 mM glycine, 29 mM sucrose and 80 ppm polysorbate 80.

FIG. 20 is the amino acid sequence of BDD-rFVIII SQ (SEQ ID NO: 3).

FIGS. 21A and 21B are the amino acid sequence of FL-rFVIII (SEQ ID NO: 1).

### DETAILED DESCRIPTION

For the purposes of interpreting this specification, the following definitions will apply, unless otherwise indicated. All references cited herein are incorporated by reference herein in their entireties.

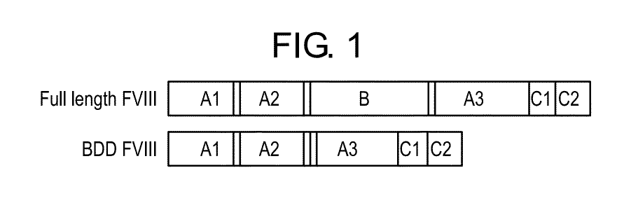

Factor VIII: Factor VIII (“FVIII”) is a coagulation factor that circulates as a heterodimer composed of a heavy chain of approximately 200 kDa and a light chain of 80 kDa. The heavy chain contains structurally related A1 and A2 domains, as well as a unique B domain, and light chain comprises the A3, C1, and C2 domains. See, e.g., Mei et al., 116 BLOOD 270-279 (2010). See also FIG. 1, showing the domains of FVIII. The term “Factor VIII” or “FVIII” as used herein refers to all Factor VIII molecules, whether derived from blood plasma or produced through the use of recombinant DNA techniques, that have some procoagulant activity characteristic of wild type human FVIII. As used herein, FVIII includes modified or truncated forms of wild type or recombinant Factor VIII that retain some or all of the procoagulant activity of wild type Factor VIII or activated wild type Factor VIII, including variants or truncated forms that have procoagulant activity exceeding the activity of wild type Factor VIII or activated wild type Factor VIII. FVIII also includes fusion products containing active Factor VIII, such as fusions with an immunoglobulin fragment or domain. Commercially available examples of therapeutic preparations containing FVIII include those sold under the trade name KOGENATE FS (available from Bayer Healthcare LLC, Berkeley, Calif., U.S.A.).

Recombinant Factor VIII: Recombinant Factor VIII (“rFVIII”) as used herein refers to FVIII that is produced using recombinant technology, or a biologically active derivative thereof, and does not include FVIII obtained from mammalian plasma.

Full-length, native human Factor VIII (“FL-FVIII”) is a 2,351 amino acid, single chain glycoprotein. The expressed 2,351 amino acid sequence is provided as SEQ. ID. NO: 1. When the expressed polypeptide is translocated into the lumen of the endoplasmic reticulum, however, a 19-amino acid signal sequence is cleaved, resulting in a second sequence. This second sequence, herein provided as SEQ. ID. NO: 2, lacks the leading 19 amino acids and is the sequence conventionally used by researchers to assign a numeric location (e.g., Arg³⁷²) to a given amino acid residue of VIII. Thus, unless specifically noted, all assignments of a numeric location of an amino acid residue as provided herein are based on SEQ. ID. NO: 2. For example, as is conventional and as used herein, when referring to mutated amino acids in BDD rFVIII, the mutated amino acid is designated by its position in the sequence of full-length FVIII. For example, a BDD rFVIII mutant can include a K1808C amino acid substitution wherein the lysine (K) at the position analogous to 1808 in the full-length sequence (here, SEQ ID NO: 2) is substituted to cysteine (C).

B-domain deleted (“BDD”) Factor VIII: As used herein, BDD or BDD-rFVIII is characterized by having an amino acid sequence with a deletion of all or part of the B-domain. In one embodiment, BDD is the molecule known as BDD-SQ, which contains a deletion of all but 14 amino acids of the B-domain of Factor VIII. In BDD-SQ, the first 4 amino acids of the B-domain (SEQ ID NO: 4) are linked to the 10 last residues of the B-domain (SEQ ID NO: 5). See, e.g., Lind et al., Novel forms of B-domain-deleted recombinant factor VIII molecules, 232 EUROPEAN JOURNAL OF BIOCHEMISTRY 19-27 (1995). See also FIG. 1 showing BDD by domain organization. BDD-SQ as used herein comprises the amino acid sequence of SEQ ID NO: 3. The B-domain of Factor VIII seems to be dispensable as a BDD molecule having a 90 kD A1-A2 heavy chain plus 80 kD light chain has been shown to be effective as a replacement therapy for hemophilia A. FVIII molecules having other portions of the B-domain deleted or all of the B-domain deleted are also included in the formulations and methods of the present invention.

BDD mutant or BDD-rFVIII mutant: BDD mutant or BDD-rFVIII mutant is a variant of BDD-SQ that maintains at least some of the FVIII procoagulant activity and differs from BDD-SQ by at least one amino acid residue. BDD mutant or BDD-rFVIII mutant includes variants that differ in amino acid sequence from BDD-SQ, for example, without limitation, by site-directed mutation of one or more amino acid residues. Without limitation, BDD mutant or BDD-rFVIII mutant includes the FVIII polypeptides with introduced cysteine residues disclosed in U.S. Pat. No. 7,928,199 (Griffin et al.).

PEGylation: PEGylation is the covalent attachment of long-chain polyethylene glycol (PEG) molecules to proteins, such as by attaching a PEG that has an active functionality that binds to a site present on FVIII. One method used for PEGylation is the attachment of a functionalized PEG moiety to lysine residues or N-terminal amines that are present in the native protein. Because FVIII contains many amine residues, amine-functionalized polymers are randomly conjugated to different sites on FVIII.

Site-directed PEGylation allows targeting of the PEG molecules to specific sites. These specific sites can include introduced surface-exposed cysteines to which the PEG polymer can be conjugated. See U.S. Pat. No. 7,632,921 (Pan et al.). PEG may also be attached to FVIII by covalent linkage to a saccharide on FVIII. See, e.g., U.S. Pat. App. Pub. 20110112028 (Turecek et al.). PEG may be attached to FVIII by enzymatic coupling of PEG to a glycan on FVIII, such as an O-glycan. Stennicke et al. disclose selective coupling of PEG to a unique O-glycan in the FVIII B-domain by incubating full-length FVIII with sialidase and excess CMP-SA-glycerol-PEG reagent in a buffer. Stennicke et al., “A novel B-domain O-glycoPEGylated FVIII (N8-GP) demonstrates full efficacy and prolonged effect in hemophilic mice models,” 121 (11) BLOOD 2108-16 (2013). U.S. Pat. App. Pub. 20130137638 (Bolt) discloses PEG attachment to a FVIII variant with a truncated B-domain. The FVIII molecule is covalently conjugated with a hydrophilic polymer via an O-linked oligosaccharide in the truncated B domain. U.S. Pat. App. Pub. 20120322738 (Behrens) discloses methods of conjugating polymers to FVIII, including covalently conjugating PEG to FVIII via an O-linked saccharide in the B-domain. As used herein, a PEGylated FVIII includes PEGylation by any method, including the various methods known in the art discussed above.

International Unit, IU: International Unit, or IU, is a unit of measurement of the blood coagulation activity (potency) of FVIII as measured by a standard assay. Standard assays include the one stage assay, as described in the art. See, e.g., Lee et al., An effect of predilution on potency assays of Factor VIII concentrates, 30 THROMBOSIS RESEARCH 511-519 (1983). The one-stage assay is based upon the activated partial thromboplastin time (aPTT). FVIII acts as a cofactor in the presence of Factor IXa, calcium, and phospholipid in the enzymatic conversion of Factor X to Xa. In this assay, the diluted test samples are incubated at 37° C. with a mixture of FVIII deficient plasma substrate and aPTT reagent. Calcium chloride is added to the incubated mixture and clotting is initiated. An inverse relationship exists between the time (seconds) it takes for a clot to form and logarithm of the concentration of FVIII:C. Activity levels for unknown samples are interpolated by comparing the clotting times of various dilutions of test material with a curve constructed from a series of dilutions of standard material of known activity and are reported in International Units per mL (IU/mL). Also useful are chromogenic assays, which may be purchased commercially, including the assay under the trade name COATEST SP FVIII (available from Chromogenix AB, Molndal, Sweden). The chromogenic assay method consists of two consecutive steps where the intensity of color is proportional to the FVIII activity. In the first step, Factor X is activated to FXa by FIXa with its cofactor, FVIIIa, in the presence of optimal amounts of calcium ions and phospholipids. Excess amounts of Factor X are present such that the rate of activation of Factor X is solely dependent on the amount of FVIII. In the second step, Factor Xa hydrolyzes the chromogenic substrate to yield a chromophore and the color intensity is read photometrically at 405 nm. Potency of an unknown is calculated and the validity of the assay is checked with the slope-ratio statistical method. Activity is reported in International Units per mL (IU/mL).

Freeze-drying, freezing, lyophilizing: “Freeze-drying,” unless otherwise indicated by the context in which it appears, shall be used to denote the portion of a lyophilization process in which the temperature of a pharmaceutical preparation is raised in the primary and secondary drying phases in order to drive water out of the preparation. The “freezing” steps of a lyophilization process are those steps which occur prior to the primary and secondary drying stages. “Lyophilizing,” unless otherwise indicated, shall refer to the entire process of lyophilization, including both the freezing steps and the freeze-drying steps.

Within certain aspects of the present disclosure, formulations comprising rFVIII and BDD-rFVIII, including formulations comprising PEGylated FVIII and BDD-rFVIII, can be lyophilized according to methodology known in the art. For example, U.S. Pat. Nos. 5,399,670 and 5,763,401 describe methodology for producing lyophilized FVIII formulations of enhanced solubility, which methodology may be employed to lyophilize the formulations described herein. The lyophilization process has a freezing phase, a primary drying phase, and a secondary drying phase. In the freezing phase, there is an annealing step. The freezing phase is performed at temperature not higher than −40° C., the annealing step occurs at temperature not higher than −15° C., the primary drying is performed at temperature not higher than 0° C., and the secondary drying is done at temperature not higher than 30° C. Once the set temperature is reached for the freezing temperature, annealing temperature, final freezing temperature, primary drying temperature, and secondary drying temperature, such temperature can be held for a reasonable time period as would be readily understood by one of skill in the art considering the particular protein sample involved, such as for one hour, two hours, three hours, or greater than three hours.

Anneal: The term “anneal” shall be used to indicate a step in the lyophilization process of a pharmaceutical preparation undergoing lyophilization, prior to the freeze-drying of the preparation, in which the temperature of the preparation is raised from a lower temperature to a higher temperature and then cooled again after a period of time.

Bulking agent: For the purposes of this application, bulking agents are those chemical entities which provide structure to the “cake” or residual solid mass of a pharmaceutical preparation after it has been lyophilized and which protect it against collapse. A crystallizable bulking agent shall mean a bulking agent as described herein which can be crystallized during lyophilization.

Surfactant: As used herein, the term “surfactant” includes “non-ionic surfactants” such as polysorbates including polysorbate 20 and polysorbate 80, polyoxamers including poloxamer 184 or 188, pluronic polyols (sold under the trade name PLURONIC, manufactured by the BASF Wyandotte Corporation), and other ethylene/polypropylene block polymers. Non-ionic surfactants stabilize the rFVIII during processing and storage by reducing interfacial interaction and prevent protein from adsorption. The use of non-ionic surfactants permits the formulations to be exposed to shear and surface stresses without causing denaturation of the rFVIII. The formulations disclosed herein include formulations having one or more non-ionic surfactant(s), exemplified herein are formulations having a polysorbate, such as polysorbate 20 (sold under the trade name TWEEN 20) or polysorbate 80 (sold under the trade name TWEEN 80).

Osmolality: As used herein, the term “osmolality” refers to a measure of solute concentration, defined as the number of osmoles of solute per kg of solvent. A desired level of osmolality can be achieved by the addition of one or more stabilizer such as a sugar or a sugar alcohol including mannitol, dextrose, glucose, trehalose, and/or sucrose. Additional stabilizers that are suitable for providing osmolality are described in references such as the handbook of Pharmaceutical Excipients (Fourth Edition, Royal Pharmaceutical Society of Great Britain, Science & Practice Publishers) or Remingtons: The Science and Practice of Pharmacy (Nineteenth Edition, Mack Publishing Company). Formulations described herein have an osmolality ranging from about 240 mOsm/kg to about 450 mOsm/kg, or about 750 mOsm/kg, or about 1000 mOsm/kg, or from about 270 mOsm/kg to about 425 mOsm/kg, or from about 300 mOsm/kg to about 410 mOsm/kg.

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.” Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to +/−10% of the unit value provided. As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term substantially is therefore used herein to capture the potential lack of completeness inherent in many biological and chemical phenomena.

The formulations of the invention described herein may be described in terms of the component concentrations by weight, such as by weight percent, or by molarity. It is to be understood that the invention also encompasses lyophilized preparations of these formulations that when reconstituted in suitable diluent, such as saline or water, for administration or storage have the concentrations reported. Ranges herein include the endpoints of the range.

Unless otherwise noted, percentage terms express weight/volume percentages and temperatures are in the Celsius scale.

### Composition Components

The FVIII compositions of the present invention may include stabilizing agents, buffering agents, sodium chloride, calcium salts, and, advantageously, other excipients. These excipients have been chosen in order to maximize the stability of FVIII in lyophilized preparations and/or in liquid preparations.

The bulking agents used in the present compositions are preferably selected from the group consisting of mannitol, glycine, and alanine. Mannitol, glycine, or alanine may be present in an amount of 1-5%, 2-3%, and 2.2-2.6%. Glycine may be the chosen bulking agent. Compositions are envisioned that do not contain a bulking agent.

The stabilizing agents used in the present compositions are selected from the group consisting of sugars or sugar alcohols, including without limitation sucrose, mannitol, dextrose, glucose and trehalose. These agents are present in the compositions in an amount of between 0.5-10%, 1-8%, 2-7%, 3-6%, 4-5%, 1-5%, 1-4%, 1-3%, or 1-2%. In compositions containing a bulking agent, sucrose is the preferred stabilizing agent in an amount of between 1-3%. In compositions lacking a bulking agent, sucrose or trehalose may be chosen as the stabilizing agent in an amount of about 8%. These sugars or sugar alcohols also function as cryo-protective agents.

In addition, buffers are present in certain of the inventive compositions. Buffers may be useful, for example, in FVIII formulations that are undergoing lyophilization, because it is believed that FVIII can be adversely affected by pH shifts during lyophilization. The buffering agents can be any physiologically acceptable chemical entity or combination of chemical entities which have the capacity to act as buffers, including histidine and MOPS (3-(N-morpholino) propanesulfonic acid). Histidine may be the chosen buffering agent in an amount of about 20 mM.

In order to preserve the activity of FVIII, the compositions of the present invention may also include calcium or another divalent cation able to interact with FVIII and maintain its activity, presumably by maintaining the association of the heavy and light chains of FVIII. Between 1 mM and 5 mM of a calcium salt can be used. The calcium salt can be calcium chloride, but can also be other calcium salts such as calcium gluconate, calcium glubionate, or calcium gluceptate. The FVIII compositions of the present invention also may include a surfactant, particularly a nonionic surfactant chosen from the group consisting of polysorbate 20 and polysorbate 80, polyoxamers including poloxamer 184 or 188, pluronic polyols (sold under the trade name PLURONIC, manufactured by the BASF Wyandotte Corporation), and other ethylene/polypropylene block polymers. The surfactant can be polysorbate 80 in an amount of about 80 ppm.

The FVIII used in the present compositions may be covalently attached to a biocompatible polymer, such as PEG. As used herein, the terms “polyethylene glycol” or “PEG” are interchangeable and include any water-soluble poly(ethylene oxide). PEG includes the following structure “—(OCH₂CH₂)ₙ—” where (n) is 2 to 4000. As used herein, PEG also includes “—CH₂CH₂—O(CH₂CH₂O)ₙ—CH₂CH₂—” and “—OCH₂CH₂)ₙO—,” depending upon whether or not the terminal oxygens have been displaced. The term “PEG” includes structures having various terminal or “end capping” groups, such as without limitation a hydroxyl or a C₁₋₂₀ alkoxy group such as methoxy. The term “PEG” also means a polymer that comprises a majority, that is to say, greater than 50%, of —OCH₂CH₂— repeating subunits. With respect to specific forms, the PEG can take any number of a variety of molecular weights, as well as structures or geometries such as branched, linear, forked, and multifunctional. As used herein, the term “PEGylation” refers to a process whereby a polyethylene glycol (PEG) is covalently attached to a molecule such as a protein. When a functional group such as a biocompatible polymer is described as activated, the functional group reacts readily with an electrophile or a nucleophile on another molecule.

The biocompatible polymer used in the conjugates disclosed herein may be any of the polymers discussed herein or known in the art. The biocompatible polymer is selected to provide the desired improvement in pharmacokinetics. For example, the identity, size and structure of the polymer is selected so as to improve the circulation half-life of FVIII or decrease the antigenicity of FVIII without an unacceptable decrease in activity. The polymer can include PEG. For example, the polymer can be a polyethylene glycol terminally capped with an end-capping moiety such as hydroxyl, alkoxy, substituted alkoxy, alkenoxy, substituted alkenoxy, alkynoxy, substituted alkynoxy, aryloxy and substituted aryloxy. In some embodiments, the polymer can include methoxypolyethylene glycol such as methoxypolyethylene glycol having a size range from 3 kD to 200 kD.

The polymer can have a reactive moiety. For example, the polymer can have a sulfhydryl reactive moiety that can react with a free cysteine on a functional FVIII polypeptide to form a covalent linkage. Such sulfhydryl reactive moieties include thiol, triflate, tresylate, aziridine, oxirane, S-pyridyl, or maleimide moieties. The polymer can be linear and include a “cap” at one terminus that is not strongly reactive towards sulfhydryls (such as methoxy) and a sulfhydryl reactive moiety at the other terminus. The conjugate can include PEG-maleimide having a size range from 5 kD to 64 kD. Alternatively, the polymer can have an amine reactive moiety such as succinimidyl propionate, succinimidyl butanoate, benzotriazole carbonate, hydroxysuccinimide, aldehyde such as propionaldehyde, butryaldehyde, acetal, piperidone, methylketone, etc. (see, e.g. U.S. Pat. No. 7,199,223 (Bossard)).

The FVIII molecule may be conjugated to a biocompatible polymer via conjugation of the polymer to the carbohydrate moieties of FVIII. See US Pat. App. Pub. 20110112028 (Turecek et al.). A FVIII molecule may be conjugated to a water-soluble polymer by conjugating a water soluble polymer to an oxidized carbohydrate moiety of FVIII. The water soluble polymer in some embodiments is selected from the group consisting of PEG, polysialic acid (“PSA”) and dextran. In still another aspect, the activated water soluble polymer is selected from the group consisting of PEG-hydrazide, PSA-hydrazine and aldehyde-activated dextran. In another aspect of the invention, the carbohydrate moiety is oxidized by incubation in a buffer comprising NaIO₄.

Suitable FVIII proteins to be used in the present invention have homology to specific known amino acid sequences. For example, suitable FVIII variants for use in the present invention are variants that have at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to known FVIII amino acid sequences, for example to the amino acid sequence of full-length FVIII (SEQ ID NO: 1) or that of BDD-FVIII (SEQ ID NO: 3). Also useful in the invention are genetic variants having defined sequence differences from a known FVIII sequence, such as FVIII molecules that comprise an amino acid sequence having 1, 2, 3, 4, 5, 6, 7, 8, 9, 10, 1-10, 1-5, 2-6 or 3-8 differences in amino acid sequence when compared to a native, known, or control sequence, such as the amino acid sequences of full-length FVIII (SEQ ID NO: 1) or BDD-SQ (SEQ ID NO: 3). Allelic variants are also useful in the present invention. Examples of allelic variants of FVIII are those disclosed in U.S. Patent Application Pub. No. 2010/0256062 (Howard et al.); Howard et al., “African-Americans Express Multiple Haplotypic Forms of the Wildtype Factor VIII (FVIII) Protein: A Possible Role for Pharmacogenetics in FVIII Inhibitor Development?” Blood, Vol. 104, 2004, Abstract 384; and Viel, K. R. et al., “Inhibitors of Factor VIII in Black Patients with Hemophilia,” The New England Journal of Medicine, Vol. 360, 2009, pp. 1618-27. Allelic variants include those with amino acid substitutions such as histidine for arginine at position 484 (R484H), glycine for arginine at position 776 (R776G), glutamic acid for aspartic acid at position 1241 (D1241E), and valine for methionine at position 2238 (M2238V). The numbering systems used to designate the amino acid substitutions are based on SEQ ID NO: 2 herein.

Methods of alignment of nucleotide and amino acid sequences for comparison are well known in the art. Alignments for the present invention may be measured using a suitable method, including by using the local homology algorithm (BESTFIT) of Smith and Waterman, Adv. Appl. Math 2:482 (1981), which may conduct optimal alignment of sequences for comparison; by using the homology alignment algorithm (GAP) of Needleman and Wunsch, J. Mol. Biol. 48:443-53 (1970); or by using the search for similarity method (Tfasta and Fasta) of Pearson and Lipman, Proc. Natl. Acad. Sci. USA 85:2444 (1988). The alignments may be performed by using computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, Gene 73:237-44 (1988); Higgins and Sharp, CABIOS 5:151-3 (1989); Corpet et al., Nucleic Acids Res. 16:10881-90 (1988); Huang et al., Computer Applications in the Biosciences 8:155-65 (1992), and Pearson et al., Meth. Mol. Biol. 24:307-31 (1994). One program to use for optimal global alignment of multiple sequences is PileUp (Feng and Doolittle, J. Mol. Evol., 25:351-60 (1987)) which is similar to the method described by Higgins and Sharp, CABIOS 5:151-53 (1989). The BLAST family of programs can be used for database similarity searches, such as for identifying other suitable FVIII molecules. See CURRENT PROTOCOLS IN MOLECULAR BIOLOGY, Chapter 19, Ausubel et al., eds., Greene Publishing and Wiley-Interscience, New York (1995).

It is believed that the B-domain of FVIII is dispensable for activity, as discussed above. In certain embodiments, the FVIII used in the invention may have all or some of the B-domain deleted. Accordingly, the present invention applies to FVIII variants or nucleotide sequences encoding such variants that comprise an amino acid sequence or encode an amino acid sequence having at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to amino acids 1-740 of the full-length FVIII (SEQ ID NO: 1) and at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to amino acids 1689-2351 of the full-length FVIII (SEQ ID NO: 1). Alternatively, the present invention applies to FVIII variants or nucleotide sequences encoding such variants that comprise an amino acid sequence or encode an amino acid sequence having at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the full-length of amino acid sequence SEQ ID NO: 1.

In certain embodiments of the invention, the FVIII may be the result of site-directed mutation, such as to create a binding site on FVIII to covalently attach a biocompatible polymer such as PEG. Site-directed mutation of a nucleotide sequence encoding polypeptide having FVIII activity may occur by any method known in the art. Methods include mutagenesis to introduce a cysteine codon at the site chosen for covalent attachment of the polymer. This may be accomplished using a commercially available site-directed mutagenesis kit such as the STRATAGENE CQUICKCHANGE II site-directed mutagenesis kit, the CLONETECH TRANSFORMER site-directed mutagenesis kit no. K1600-1, the INVITROGEN GENTAYLOR site-directed mutagenesis system no. 12397014, the PROMEGA ALTERED SITES II in vitro mutagenesis system kit no. Q6210, or the TAKARA MIRUS BIO LA PCR mutagenesis kit no. TAK RR016. Conjugates described herein may be prepared by first replacing the codon for one or more amino acids on the surface of the functional FVIII polypeptide with a codon for cysteine, producing the cysteine mutant in a recombinant expression system, reacting the mutant with a cysteine-specific polymer reagent, and purifying the mutein. In this system, the addition of a polymer at the cysteine site can be accomplished through a maleimide active functionality on the polymer. See, e.g., U.S. Pat. No. 7,632,921 (Pan et al.).

The amount of sulfhydryl reactive polymer used should be at least equimolar to the molar amount of cysteines to be derivatized and can be present in excess. A 5-fold or a 10-fold molar excess of sulfhydryl reactive polymer can be used. Other conditions useful for covalent attachment are within the skill of those in the art.

The predefined site for covalent binding of the polymer, e.g., PEG, can be selected from sites exposed on the surface of the rFVIII or BDD rFVIII polypeptide that are not involved in FVIII activity or involved in other mechanisms that stabilize FVIII in vivo, such as binding to vWF. Such sites are also best selected from those sites known to be involved in mechanisms by which FVIII is deactivated or cleared from circulation. Sites for substituting an amino acid with a cysteine include an amino acid residue in or near a binding site for (a) low density lipoprotein receptor related protein, (b) a heparin sulphate proteoglycan, (c) low density lipoprotein receptor and/or (d) FVIII inhibitory antibodies. By “in or near a binding site” means a residue that is sufficiently close to a binding site such that covalent attachment of a biocompatible polymer to the site would result in steric hindrance of the binding site. Such a site is expected to be within 20 Å of a binding site, for example.

The biocompatible polymer can be covalently attached to the rFVIII or BDD rFVIII polypeptide, or mutant variant thereof, at one or more of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284. One or more sites, such as one or two, on the functional FVIII polypeptide may be the predefined sites for polymer attachment. In particular embodiments, the polypeptide is mono-PEGylated or diPEGylated, meaning one PEG or two PEG molecules are attached to each FVIII, respectively.

Site directed PEGylation of a FVIII mutant can also be achieved by: (a) expressing a site-directed FVIII mutant wherein the mutant has a cysteine replacement for an amino acid residue on the exposed surface of the FVIII mutant and that cysteine is capped; (b) contacting the cysteine mutant with a reductant under conditions to mildly reduce the cysteine mutant and to release the cap; (c) removing the cap and the reductant from the cysteine mutant; and (d) after the removal of the reductant, treating the cysteine mutant with PEG comprising a sulfhydryl coupling moiety under conditions such that PEGylated FVIII mutein is produced. The sulfhydryl coupling moiety of the PEG is selected from the group consisting of thiol, triflate, tresylate, aziridine, oxirane, S-pyridyl and maleimide moieties, and can be maleimide.

In another embodiment a biocompatible polymer such as, e.g., PEG, is covalently attached through use of a polymer functionalized with an amine-specific functional group. The polymer may be functionalized with, for example, mPEG tresylate or mPEG succinimidyl succinate such that it is reactive at lysines on FVIII. The coupling can occur at random lysines on FVIII by adding activated mPEG in a solid state to a solution of FVIII and rotating at room temperature. The degree of modification may be loosely controlled by the level of excess activated mPEG used. Rostin et al., “B-Domain Deleted Recombinant Coagulation Factor VIII Modified with Monomethoxy Polyethylene Glycol,” 11 Bioconjug. Chem., 2000, pp. 387-396. Further examples of PEGylation conditions and reagents are provided in U.S. Pat. No. 7,199,223 (Bossard) and U.S. Pat. No. 4,970,300 (Fulton). The present invention is also directed to methods for covalently attaching a biocompatible polymer to FVIII in which one of the liquid formulations of the invention is the solution in which the reaction occurs.

The present disclosure also provides methods for the treatment of hemophilia A in a patient, comprising the administration to the patient in need thereof a therapeutically effective amount of one or more formulations described herein. These formulations may be administrated to a patient via intravenous injection, subcutaneous injection, or through continuous infusion.

As used herein, the term “therapeutically effective amount” of a rFVIII formulation or a PEGylated rFVIII formulation refers to an amount of the formulation that provides therapeutic effect in an administration regimen to a patient in need thereof. For example, for replacement therapy for hemophilia A, an amount of between 10-30 IU/kg body weight of recombinant full-length FVIII for intravenous injection is recommended. For prophylaxis in a child with hemophilia A, 25 IU/kg body weight of recombinant full-length FVIII for intravenous injection is recommended. Prior to surgery, 15-30 IU/kg (minor surgery) or 50 IU/kg (major surgery) of recombinant full-length FVIII for intravenous injection is recommended for a child with hemophilia A. Corresponding dosages for the various FVIII molecules used in the formulations of the invention can be determined by those of skill in the art. Preferably the therapeutic FVIII formulations of the invention are provided in single use dosages of 100, 250, 300, 400, 500, 600, 700, 800, 900, 1000, 1200, 1400, 1600, 1800, 2000, 2200, 2400, 2600, 2800, 3000, 3200, 3400, 3600, 3800, 4000, or 5000 IU, or in a range between any two of these dosages, i.e., in a range of from 100 to 250 IU, from 100 to 500 IU, from 1000 to 2000 IU, etc., inclusive of the endpoints. Because of their low viscosity, the presently disclosed rFVIII and PEG-rFVIII formulations can be conveniently processed via, for example, ultrafiltration and sterile filtration and can be administered to a patient via injection, including intravenous injection, subcutaneous injection, and continuous infusion.

The FVIII compositions described in this application can be lyophilized and reconstituted in the indicated concentrations. These FVIII compositions can also be reconstituted in more dilute form. For example, a preparation according the present invention which is lyophilized and/or normally reconstituted in 2 ml of solution can also be reconstituted in a larger volume of diluent, such as 5 ml. This is particularly appropriate when the FVIII preparation is being injected into a patient immediately, since in this case the FVIII is less likely to lose activity, which may occur more rapidly in more dilute solutions of FVIII.

### Embodiment 1

Recombinant FVIII is produced in the absence of plasma proteins that stabilize plasma-derived FVIII, such as von Willebrand factor (vWF). The absence of such stabilizing proteins makes rFVIII extremely labile. In addition, rFVIII is present at very low concentrations in therapeutic solutions (0.02 mg protein per ml for a therapeutic dose of 1000 IU BDD-SQ), which makes surface adsorption a cause for loss of activity.

One embodiment of the invention is a formulation of rFVIII, particularly BDD-rFVIII, and even more particularly BDD-rFVIII mutants with cross-linking between the domains, such as between the A1 and A2 or A3 domains. In one embodiment the formulation is of a FVIII having double cysteine mutations which cross-link the A2 and the A1 or the A3 domains, preferably the A2 and the A3 domains, such as through disulfide bridges as described in U.S. Pat. No. 7,928,199 to Griffin et al. (issued Apr. 19, 2011), including without limitation mutants of FVIII, including mutants of BDD SQ (SEQ ID NO: 3), in which one or more cysteines have been introduced at one or more sites; such that at least one pair of cysteines creates a disulfide bond not found in wild type FVIII. In one embodiment, the mutant FVIII comprises at least one pair of recombinantly introduced cysteines, wherein the pair of cysteines replaces a pair of residues selected from the group consisting of Met 662 and Asp 1828, Ser 268 and Phe 673, Ile 312 and Pro 672, Ser 313 and Ala 644, Met 662 and Lys 1827, Tyr 664 and Thr 1826, Pro 264 and Gln 645, Arg 282 and Thr 522, Ser 285 and Phe 673, His 311 and Phe 673, Ser 314 and Ala 644, Ser 314 and Gln 645, Val 663 and Glu 1829, Asn 694 and Pro 1980, and Ser 695 and Glu 1844. Suitable FVIII molecules for the formulations of the present embodiment suffer the disadvantage of aggregating in solution and/or show a high propensity for precipitation. These disadvantages create problems preparing a stable therapeutic dosage. Also, if the FVIII molecules are to be further processed, such as by covalent attachment of a biocompatible polymer such as PEG, the FVIII molecules are preferably in solution to provide good processing, such as good yields upon PEGylation, which requires that the FVIII be in suspension or solution and not be aggregated. In one embodiment, the FVIII formulations of the present application contain sodium chloride or potassium chloride in an amount sufficient to reduce or abolish precipitation and/or aggregation and to provide stability.

Formulations of Embodiment 1 may be as follows. A rFVIII formulation comprising:
(a) a range of from about 0 mM to about 20 mM, from about 1 mM to about 20 mM, from about 1 mM to about 50 mM, from about 10 mM to about 50 mM, from about 10 mM to about 20 mM, from about 10 mM to about 30 mM, or from about 20 mM to about 50 mM histidine;
(b) a range of from about 0 mM to about 29 mM, from about 1 mM to about 29 mM, from about 1 mM to about 300 mM, from about 10 mM to about 30 mM, from about 10 mM to about 100 mM, from about 10 mM to about 200 mM, from about 10 mM to about 50 mM, from about 29 mM to about 58 mM, from about 34 mM to about 58 mM, from about 58 mM to about 100 mM, or from about 100 mM to about 300 mM, or an amount of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, or about 300 mM of a sugar or sugar alcohol;
(c) a range of from about 1 mM to about 2 mM, from about 1 mM to about 2.5 mM, from about 1.5 mM to about 3.5 mM, or from about 1 mM to about 5 mM divalent cation such as a divalent calcium salt, including calcium chloride;
(d) a range of from about 150 mM to about 250 mM, from about 150 mM to about 220 mM, from about 150 mM to about 200 mM, from about 150 mM to about 190 mM, from about 170 mM to about 250 mM; from about 200 mM to about 220 mM, from about 170 mM to about 200 mM, from about 200 mM to about 250 mM, from about 170 mM to about 220 mM, from about 190 mM to about 220 mM, from about 210 mM to about 220 mM, from about 150 mM to about 180 mM, from about 150 mM to about 160 mM, or from about 220 mM to about 250 mM sodium chloride or potassium chloride;
(e) a range of from about 20 ppm to about 200 ppm, from about 20 ppm to about 50 ppm, from about 20 ppm to about 80 ppm, from about 50 ppm to about 80 ppm, from about 80 ppm to about 100 ppm, from about 80 ppm to about 200 ppm, from about 50 ppm to about 100 ppm, or from about 50 ppm to about 200 ppm of a non-ionic surfactant, or about 60, 65, 70, 75, 80, 85, 90, 95, 100, 105, 110, 115, 120, 125, 130, 135, 140, 145, 150, 160, 170, 180, 190, 200, or 210 ppm of a non-ionic surfactant;
(f) a range of from about 0 mM to about 50 mM, from about 1 mM to about 50 mM, from about 50 mM to about 100 mM, from about 100 mM to about 150 mM, from about 150 mM to about 293 mM, from about 150 mM to about 400 mM, from about 200 mM to about 300 mM; from about 250 mM to about 300 mM, or from about 200 mM to about 400 mM glycine, or about 100, 200, 210, 230, 240, 250, 260, 270, 280, 290, 293, 295, 300, 310, 320, 330, 340, 350, 360, 370, 380, 390, or about 400 mM glycine; and
(g) a range of from about 100 IU/ml to about 5000 IU/ml, from about 100 IU/ml to about 2000 IU/ml, from about 100 IU/ml to about 3000 IU/ml, from about 100 IU/ml to about 4000 IU/ml, from about 100 IU/ml to about 1200 IU/ml, from about 250 IU/ml to about 5000 IU/ml, from about 250 IU/ml to about 1000 IU/ml, from about 250 IU/ml to about 2000 IU/ml, from about 250 IU/ml to about 3000 IU/ml, from about 500 IU/ml to about 1000 IU/ml, from about 500 IU/ml to about 3000 IU/ml, from about 1000 IU/ml to about 2000 IU/ml, from about 1000 IU/ml to about 3000 IU/ml, from about 1000 IU/ml to about 4000 IU/ml, or from about 1000 IU/ml to about 5000 IU/ml, or about 100, 200, 300, 400, 500, 600, 700, 800, 900, 1000, 1200, 1400, 1600, 1800, 2000, 2200, 2400, 2600, 2800, 3000, 3200, 3500, 3800, 4000, 4200, 4500, 4800, 5000, 5500, or 6000 IU/ml of a rFVIII selected from rFVIII, BDD-rFVIII, BDD-rFVIII mutants, and BDD-rFVIII mutants with cross-linking between FVIII domains,
   wherein the rFVIII formulation has a pH in a range of from about pH 6.0 to about pH 6.5, from about pH 6.0 to about pH 7.0, from about pH 6.0 to about pH 7.5, from about pH 6.5 to about pH 7.5, or from about pH 7.0 to about pH 7.5, or a pH of about pH 6.0, 6.5, 7.0, 7.1, 7.2, 7.3, 7.4 or about pH 7.5.

In another version of Embodiment 1, the invention pertains to a rFVIII formulation comprising:
(a) a range of from about 0 mM to about 20 mM, from about 10 mM to about 50 mM, or from about 10 mM to about 30 mM histidine;
(b) a range of from about 1 mM to about 29 mM, from about 10 mM to about 30 mM, from about 10 mM to about 100 mM, from about 10 mM to about 200 mM, from about 10 mM to about 50 mM, from about 29 mM to about 58 mM, or from about 34 mM to about 58 mM, or an amount of about 25, 26, 27, 28, 29, 30, 31, 32, 33, 34, 35, 36, 37, 38, 39, or 40 mM of a sugar or sugar alcohol;
(c) a range of from about 1 mM to about 2 mM, from about 1 mM to about 2.5 mM, from about 1.5 mM to about 3.5 mM, or from about 1 mM to about 5 mM divalent cation such as a divalent calcium salt, including calcium chloride;
(d) a range of from about 150 mM to about 200 mM, from about 150 mM to about 220 mM, from about 170 mM to about 250 mM sodium chloride or potassium chloride;
(e) a range of from about 20 ppm to about 80 ppm, from about 80 ppm to about 100 ppm, from about 50 ppm to about 100 ppm, or from about 50 ppm to about 200 ppm of a non-ionic surfactant, or about 70, 75, 80, 85, 90, 95, 100, 105, 110, 115, or 120 ppm of a non-ionic surfactant;
(f) a range of from about 1 mM to about 50 mM, from about 150 mM to about 300 mM, from about 150 mM to about 400 mM, from about 200 mM to about 300 mM; or from about 250 mM to about 300 mM, or about 250, 260, 270, 280, 290, 293, 295, 300, 310, or about 320 mM glycine; and
(g) a range of from about 100 IU/ml to about 5000 IU/ml, from about 100 IU/ml to about 2000 IU/ml, from about 100 IU/ml to about 3000 IU/ml, from about 100 IU/ml to about 4000 IU/ml, from about 100 IU/ml to about 1200 IU/ml, from about 250 IU/ml to about 5000 IU/ml, from about 250 IU/ml to about 1000 IU/ml, from about 250 IU/ml to about 2000 IU/ml, from about 250 IU/ml to about 3000 IU/ml, from about 500 IU/ml to about 1000 IU/ml, from about 500 IU/ml to about 3000 IU/ml, from about 1000 IU/ml to about 2000 IU/ml, from about 1000 IU/ml to about 3000 IU/ml, from about 1000 IU/ml to about 4000 IU/ml, or from about 1000 IU/ml to about 5000 IU/ml, or about 100, 200, 300, 400, 500, 600, 700, 800, 900, 1000, 1200, 1400, 1600, 1800, 2000, 2200, 2400, 2600, 2800, 3000, 3200, 3500, 3800, 4000, 4200, 4500, 4600, 4800, 5000, 5500, or 6000 IU/ml of a rFVIII selected from rFVIII, BDD-rFVIII, BDD-rFVIII mutants, and BDD-rFVIII mutants with cross-linking between FVIII domains,
   wherein the rFVIII formulation has a pH in a range of from about pH 6.0 to about pH 7.5, from about pH 6.5 to about pH 7.5, or from about pH 7.0 to about pH 7.5, or a pH of about pH 6.0, 6.5, 7.0, 7.1, 7.2, 7.3, 7.4 or about pH 7.5.

In certain embodiments the sugar or sugar alcohol is sucrose and sodium chloride is present.

In another version of Embodiment 1, the invention pertains to a rFVIII formulation comprising:
(a) a range of from about 10 mM to about 30 mM histidine;
(b) a range of from about 10 mM to about 30 mM, from about 10 mM to about 100 mM, from about 10 mM to about 200 mM, from about 10 mM to about 50 mM, from about 29 mM to about 58 mM, or from about 34 mM to about 58 mM of a sugar or sugar alcohol;
(c) a range of from about 1 mM to about 2 mM, from about 1 mM to about 2.5 mM, from about 1.5 mM to about 3.5 mM, or from about 1 mM to about 5 mM of a divalent calcium salt, including calcium chloride;
(d) a range of from about 150 mM to about 220 mM, from about 170 mM to about 250 mM sodium chloride or potassium chloride;
(e) a range of from about 50 ppm to about 200 ppm of a non-ionic surfactant;
(f) a range of from about 1 mM to about 50 mM, from about 150 mM to about 300 mM, from about 150 mM to about 400 mM, from about 200 mM to about 300 mM; or from about 250 mM to about 300 mM glycine; and
(g) a range of from about 100 IU/ml to about 5000 IU/ml, from about 100 IU/ml to about 2000 IU/ml, from about 100 IU/ml to about 3000 IU/ml, from about 100 IU/ml to about 4000 IU/ml, from about 100 IU/ml to about 1200 IU/ml, from about 250 IU/ml to about 5000 IU/ml, from about 250 IU/ml to about 1000 IU/ml, from about 250 IU/ml to about 2000 IU/ml, from about 250 IU/ml to about 3000 IU/ml, from about 500 IU/ml to about 1000 IU/ml, from about 500 IU/ml to about 3000 IU/ml, from about 1000 IU/ml to about 2000 IU/ml, from about 1000 IU/ml to about 3000 IU/ml, from about 1000 IU/ml to about 4000 IU/ml, or from about 1000 IU/ml to about 5000 IU/ml, or about 100, 200, 300, 400, 500, 600, 700, 800, 900, 1000, 1200, 1400, 1600, 1800, 2000, 2200, 2400, 2600, 2800, 3000, 3200, 3500, 3800, 4000, 4200, 4500, 4600, 4800, 5000, 5500, or 6000 IU/ml of a rFVIII selected from rFVIII, BDD-rFVIII, BDD-rFVIII mutants, and BDD-rFVIII mutants with cross-linking between FVIII domains, wherein the rFVIII formulation has a pH in a range of from about pH 6.0 to about pH 7.5, from about pH 6.5 to about pH 7.5, or from about pH 7.0 to about pH 7.5, or a pH of about pH 6.0, 6.5, 7.0, 7.1, 7.2, 7.3, 7.4 or about pH 7.5.

In yet another version of Embodiment 1, the invention pertains to a rFVIII formulation comprising:
(a) a range of from about 10 mM to about 30 mM histidine;
(b) a range of from about 10 mM to about 50 mM of sucrose;
(c) a range of from about 1.5 mM to about 3.5 mM calcium chloride;
(d) a range of from about 150 mM to about 220 mM or from about 170 mM to about 220 mM sodium chloride;
(e) a range of from about 70 ppm to about 90 ppm of a non-ionic surfactant;
(f) a range of from about 200 mM to about 300 mM or from about 250 mM to about 300 mM glycine; and
(g) a range of from about 100 IU/ml to about 2000 IU/ml, from about 100 IU/ml to about 3000 IU/ml, from about 250 IU/ml to about 1000 IU/ml, from about 250 IU/ml to about 2000 IU/ml, from about 250 IU/ml to about 3000 IU/ml, from about 500 IU/ml to about 1000 IU/ml, from about 500 IU/ml to about 3000 IU/ml, from about 1000 IU/ml to about 2000 IU/ml, or from about 1000 IU/ml to about 3000 IU/ml of a rFVIII selected from BDD-rFVIII, BDD-rFVIII mutants, and BDD-rFVIII mutants with cross-linking between FVIII domains, wherein the rFVIII formulation has a pH in a range of from about pH 6.0 to about pH 7.5, from about pH 6.5 to about pH 7.5, or from about pH 7.0 to about pH 7.5, or a pH of about pH 6.0, 6.5, 7.0, 7.1, 7.2, 7.3, 7.4 or about pH 7.5.

The rFVIII formulations of embodiment 1 may optionally contain albumin, such as HSA. In certain embodiments, HSA is present at a range of from about 10 to about 50 mg/mL, from about 15 to about 30 mg/mL, from about 20 to about 30 mg/mL or from about 25 to about 30 mg/mL.

### Embodiment 2

During covalent addition of a biocompatible polymer to FVIII it was observed that buffer components may interfere with the covalent addition. For example, when FVIII was covalently coupled to PEG using PEG functionalized to have an amine-reactive group that would add at lysine residues, amine-containing components in the reaction buffer were observed to interfere with the reaction. Accordingly, the present invention includes improved liquid FVIII formulations or buffers in which the polymer addition reaction to FVIII may occur. In one version of Embodiment 2, the liquid FVIII formulations do not comprise, or comprises less than 10% by weight, or less than 5% by weight, or less than 1% by weight, or less than 0.5% by weight or only a trace amount of components with primary or secondary amine groups, other than FVIII. The inventive FVIII formulations of this embodiment include formulations that avoid the use of histidine and glycine. Histidine and glycine contain amines that may interfere with the PEGylation process.

One version of Embodiment 2 of the invention is a formulation of rFVIII having buffer capacity at pH 6-7 that does not form an insoluble complex or chelate with calcium chloride (an important rFVIII stabilizer) and does not contain components with primary or secondary amine groups, or contains such components at a weight percent of 10% or less, 5% or less, 1% or less, or in trace amounts. This formulation may include MOPS in a range of from 10 mM to 100 mM, in a range of from 10 mM to 70 mM, in a range of from 10 mM to 50 mM, in a range of from 10 mM to 40 mM, in a range of from 10 mM to 30 mM, in a range of from 12 mM to 30 mM, in a range of from 14 mM to 30 mM, in a range of from 16 mM to 30 mM, in a range of from 18 mM to 30 mM, in a range of from 20 mM to 28 mM, in a range of from 12 mM to 28 mM, in a range of from 12 mM to 26 mM, in a range of from 12 mM to 24 mM, in a range of from 12 mM to 22 mM, in a range of from 14 mM to 22 mM, or in a range of from 18 mM to 22 mM, or may contain about 10, 11, 12, 13, 14, 15, 16, 17, 18, 19, 20, 21, 22, 23, 24, 25, 26, 27, 28, 29, or 30 mM MOPS. This formulation includes rFVIII in a range of from about 100 IU/ml to about 1000 IU/ml, from about 100 IU/ml to about 500 IU/ml, from about 100 IU/ml to about 2000 IU/ml, from about 100 IU/ml to about 3000 IU/ml, from about 500 IU/ml to about 3000 IU/ml, from about 500 IU/ml to about 2000 IU/ml, from about 500 IU/ml to about 2500 IU/ml, from about 500 IU/ml to about 1200 IU/ml, from about 500 IU/ml to about 1000 IU/ml, from about 500 IU/ml to about 1500 IU/ml, from about 1000 IU/ml to about 2000 IU/ml, from about 1000 IU/ml to about 3000 IU/ml, from about 1000 IU/ml to about 2500 IU/ml, from about 1000 IU/ml to about 1500 IU/ml, from about 1000 IU/ml to about 6000 IU/ml, or from about 1000 IU/ml to about 5000 IU/ml or about 100, 200, 300, 400, 500, 600, 700, 800, 900, 1000, 1200, 1400, 1600, 1800, 2000, 2200, 2400, 2600, 2800, 3000, 3200, 3500, 3800, 4000, 4200, 4500, 4800, 5000, 5500, or 6000 IU/ml of rFVIII. It is also possible that the invention may be used with rFVIII formulations having higher activity than 6000 IU/ml.

In one version of Embodiment 2, the rFVIII formulation comprises FVIII or BDD that is recombinantly produced. In another version of Embodiment 2, the formulation comprises recombinantly produced full-length FVIII, such as FVIII comprising the amino acid sequence of SEQ ID NO: 1 or an allelic variant thereof. In another version of Embodiment 2, the formulation comprises a mutant of BDD or a mutant of FL-FVIII.

This formulation may also include a sugar or a sugar alcohol such as sucrose in a range of from 0.5% to 10%, in a range of from 0.6% to 10%, in a range of from 0.7% to 10%, in a range of from 0.8% to 10%, in a range of from 0.9% to 10%, in a range of from 1.0% to 10%, in a range of from 0.6% to 5%, in a range of from 0.6% to 2.5%, in a range of from 0.6% to 2.0%, in a range of from 0.6% to 1.5%, in a range of from 0.6% to 1.2%, in a range of from 0.8% to 1.2%, in a range of from 0.9% to 1.2%, or in a range of from 0.9% to 1.1% by weight, or at about 0.8, 0.9, 1.0, 1.1, 1.2, 1.3, 1.4, 1.5, 1.6, 1.7, 1.8, 1.9, or 2.0% by weight. This formulation may also include a divalent cation such as a calcium salt, such as calcium chloride, in a range of from 0.5 mM to 20 mM, in a range of from 1 mM to 10 mM, in a range of from 1 mM to 5 mM, in a range of from 1.5 mM to 5 mM, in a range of from 2 mM to 5 mM, in a range of from 2.5 mM to 5 mM, in a range of from 3 mM to 5 mM, in a range of from 3.5 mM to 5 mM, in a range of from 4 mM to 5 mM, in a range of from 1.5 mM to 4.5 mM, in a range of from 1.5 mM to 4 mM, in a range of from 1.5 mM to 3.5 mM, in a range of from 1.5 mM to 3 mM, in a range of from 1.5 mM to 2.5 mM, in a range of from 2 mM to 3 mM, in a range of from 2.2 mM to 2.8 mM, or in a range of from 2.4 mM to 2.6 mM. This formulation may also include sodium chloride or potassium chloride in a range of from 10 mM to 100 mM, in a range of from 10 mM to 70 mM, in a range of from 10 mM to 50 mM, in a range of from 15 mM to 50 mM, in a range of from 20 mM to 50 mM, in a range of from 25 mM to 50 mM, in a range of from 30 mM to 50 mM, in a range of from 15 mM to 45 mM, in a range of from 15 mM to 40 mM, in a range of from 15 mM to 35 mM, in a range of from 20 mM to 45 mM, in a range of from 20 mM to 40 mM, in a range of from 25 mM to 40 mM, in a range of from 25 mM to 35 mM, in a range of from 25 mM to 30 mM, or in a range of from 30 mM to 35 mM. This formulation may also include a non-ionic surfactant such as polysorbate 20 or polysorbate 80 in a range of from 50 to 150 ppm, in a range of from 60 ppm to 150 ppm, in a range of from 70 ppm to 150 ppm, in a range of from 80 ppm to 150 ppm, in a range of from 60 ppm to 140 ppm, in a range of from 60 ppm to 130 ppm, in a range of from 60 ppm to 120 ppm, in a range of from 60 ppm to 110 ppm, in a range of from 60 ppm to 100 ppm, in a range of from 60 ppm to 90 ppm, in a range of from 70 ppm to 90 ppm, in a range of from 70 ppm to 80 ppm, and in a range of from 80 ppm to 90 ppm. This composition provides acceptable stability to rFVIII in solution, and can be used as a reaction buffer during the conjugation of a polymer to FVIII using a polymer functionalized to be active at amine residues.

In one version of Embodiment 2, the invention is related to a rFVIII formulation comprising
(a) MOPS in a range of from 12 mM to 28 mM, in a range of from 12 mM to 22 mM, or in a range of from 18 mM to 22 mM;
(b) FVIII in a range of from 100 IU/ml to 3000 IU/ml, or in a range of from 1000-1500 IU/ml;
(c) sucrose in a range of from 0.5% to 5%, in a range of from 0.6% to 2.5%, or in a range of from 0.9% to 1.1%;
(d) sodium chloride or potassium chloride in a range of from 10 mM to 50 mM, in a range of from 15 mM to 35 mM, or in a range of from 25 mM to 35 mM;
(e) a divalent calcium salt, such as calcium chloride, in a range of from 1 mM to 5 mM, in a range of from 1.5 mM to 3.5 mM, or in a range of from 2.4 mM to 2.6 mM; and
(f) non-ionic surfactant such as polysorbate 20 or polysorbate 80 in a range of from 60 ppm to 100 ppm, or in a range of from 70 ppm to 90 ppm;
   wherein the rFVIII formulation contains less than 10%, less than 5%, less than 1%, less than 0.5%, or less than a trace level, or is essentially free, of a component having a primary or secondary amine group.

The invention also is directed to a method of conjugating an amine-reactive biocompatible polymer, such as an amine-reactive PEG, to FVIII comprising suspending or dissolving the FVIII in a rFVIII formulation of Embodiment 2, adding the amine-reactive polymer, and incubating the resulting mixture under conditions of time and temperature such that conjugation occurs. Such conditions preferably are at about ambient temperature. The polymer may be added at excess molar amounts (1-100-fold excess) over the FVIII. The polymer and FVIII may be conjugated by incubation together for several hours with rotation or stirring.

Although the above formulations of Embodiment 2 have been shown to be useful as reaction buffers during polymer addition involving amine-reactive functional groups, it is envisioned that the formulations are also useful in other contexts outside of such reactions and therefore that the formulations may be used when stable FVIII formulations are required.

### Embodiment 3

In Embodiment 3, the rFVIII formulations comprise NaCl, MOPS, a divalent calcium ion or another divalent cation, and optionally a nonionic surfactant and/or optionally a sugar or a sugar alcohol. The formulations of Embodiment 3 in particular are shown to provide storage without aggregation of FVIII molecules that are not conjugated to a biocompatible polymer, such as FVIII not covalently attached to PEG and not covalently attached to any polymer other than glycans present in wild-type FVIII. The formulations of Embodiment 3 are particularly suitable for non-PEGylated BDD. As used herein, “nonconjugated FVIII” refers to FVIII that is not conjugated to a polymer other than to a glycan associated with a native mammalian glycosylation pattern resulting from the host cell in which the FVIII is produced. For example, “nonconjugated FVIII” includes wild type human FVIII that is recombinantly produced in a mammalian host cell such as a BHK cell or a CHO cell such as the marketed products KOGENATE® and RECOMBINATE® FVIII.

One version of Embodiment 3 of the compositions described herein is a composition that provides stability for FVIII and contains sodium chloride in a range of from 150 mM to 300 mM, from 150 mM to 275 mM, from 150 mM to 250 mM, from 150 mM to 225 mM, from 150 mM to 200 mM, from 150 mM to 175 mM, from 175 mM to 300 mM, from 175 mM to 275 mM, from 175 mM to 250 mM, from 175 mM to 225 mM, from 175 mM to 200 mM, from 175 mM to 190 mM; from 200 mM to 300 mM, from 200 mM to 275 mM, from 200 mM to 250 mM, from 200 mM to 225 mM, from 200 mM to 210 mM, from 250 mM to 300 mM; or about 150, 160, 170, 180, 190, 200, 210, 220, 230, 240, 250, 260, 270, 280, 290, or 300 mM. The compositions also include MOPS buffer in a range of from 10 mM to 100 mM, in a range of from 10 mM to 60 mM, in a range of from 10 mM to 50 mM, in a range of from 10 mM to 40 mM, in a range of from 10 mM to 30 mM, in a range of from 12 mM to 30 mM, in a range of from 14 mM to 30 mM, in a range of from 16 mM to 30 mM, in a range of from 18 mM to 30 mM, in a range of from 12 mM to 28 mM, in a range of from 12 mM to 26 mM, in a range of from 12 mM to 24 mM, in a range of from 16 mM to 24 mM, in a range of from 18 mM to 24 mM, in a range of from 20 mM to 24 mM, or in a range of from 18 mM to 22 mM. The compositions also include a divalent cation such as calcium chloride in a range of from 1 mM to 20 mM, in a range of from 5 mM to 10 mM, in a range of from 1 mM to 30 mM, in a range of from 6 mM to 30 mM, in a range of from 7 mM to 30 mM, in a range of from 8 mM to 30 mM, in a range of from 5 mM to 20 mM, in a range of from 5 mM to 25 mM, or in a range of from 9 mM to 12 mM. The amount of rFVIII present in the formulations of Embodiment 3 may be the same as the amount provided in Embodiment 1.

The compositions may also include a sugar or sugar alcohol such as sucrose in a range of from 0.5% to 10%, in a range of from 0.6% to 10%, in a range of from 0.7% to 10%, in a range of from 0.8% to 10%, in a range of from 0.9% to 10%, in a range of from 1.0% to 10%, in a range of from 0.5% to 5%, in a range of from 0.6% to 5%, in a range of from 0.7% to 5%, in a range of from 0.8% to 5%, in a range of from 0.9% to 5%, in a range of from 1.0% to 5%, in a range of from 0.5% to 2.5%, in a range of from 0.6% to 2.5%, in a range of from 0.5% to 2.0%, in a range of from 0.5% to 1.5%, in a range of from 0.6% to 1.2%, in a range of from 0.8% to 1.2%, in a range of from 0.9% to 1.2%, or in a range of from 0.9% to 1.1%. The compositions may also include a non-ionic surfactant such as polysorbate 80 in a range of from 20 ppm to 250 ppm, in a range of from 50 ppm to 250 ppm, in a range of from 50 ppm to 150 ppm, in a range of from 60 ppm to 150 ppm, in a range of from 70 ppm to 150 ppm, in a range of from 80 ppm to 150 ppm, in a range of from 60 ppm to 140 ppm, in a range of from 60 ppm to 130 ppm, in a range of from 60 ppm to 120 ppm, in a range of from 60 ppm to 110 ppm, in a range of from 60 ppm to 100 ppm, in a range of from 70 ppm to 110 ppm, in a range of from 70 ppm to 105 ppm, in a range of from 70 ppm to 100 ppm, in a range of from 80 ppm to 100 ppm, or in a range of from 90 ppm to 110 ppm.

In certain versions of Embodiment 3 the FVIII formulation is free of histidine and/or (4-(2-hydroxyethyl)-1-piperazineethanesulfonic acid) (“HEPES”) and/or albumin, or contains less than 0.1%, less than 0.5%, less than 0.8%, less than 1.0%, or less than 5.0% by weight of histidine, and/or HEPES, and/or albumin. One version of Embodiment 3 is a FVIII formulation essentially free of histidine, HEPES and albumin.

### Embodiment 4

Polymer-conjugated FVIII, such as PEGylated FVIII, may be more hydrophilic than the corresponding unconjugated FVIII. Accordingly, formulations for conjugated FVIII such as PEGylated FVIII may require different components than those identified for unconjugated FVIII. Applicants prepared a PEGylated FVIII in a buffer that contained elevated levels of NaCl (200 mM). Such elevated levels of sodium chloride were observed to impose difficulties during lyophilization. Applicants discovered compositions of the present invention for polymer-conjugated FVIII that avoid undesirably high levels of NaCl, avoid the formation of aggregates and substantially retain potency of FVIII when stored over six days at ambient temperature. The present application provides the unexpected result that sodium chloride concentration can be reduced from 200 mM to 50 mM and still achieve potency of the rFVIII after storage at ambient temperature. In Embodiment 4, the rFVIII formulations comprise a buffer such as histidine or MOPS, NaCl, a divalent calcium ion or another divalent cation, and optionally a nonionic surfactant and/or optionally a sugar or a sugar alcohol. The formulations of Embodiment 4 in particular are shown to provide storage without aggregation of FVIII molecules that are conjugated to a biocompatible polymer, particularly a hydrophilic biocompatible polymer such as PEG. As used herein, “conjugated FVIII” refers to FVIII that is conjugated to a polymer other than to a glycan associated with a native mammalian glycosylation pattern resulting from the host cell in which the FVIII is produced.

One version of Embodiment 4 described herein is a rFVIII composition that contains sodium chloride in a range of from 25 mM to 200 mM, in a range of from 25 mM to 175 mM, in a range of from 25 mM to 150 mM, in a range of from 25 mM to 125 mM, in a range of from 25 mM to 100 mM, in a range of from 25 mM to 75 mM, in a range of from 25 mM to 50 mM, in a range of from 40 mM to 55 mM, in a range of from 25 mM to 35 mM, in a range of from 25 mM to 30 mM, in a range of from 30 mM to 60 mM, in a range of from 50 mM to 200 mM, in a range of from 50 mM to 175 mM, in a range of from 50 mM to 150 mM, in a range of from 50 mM to 125 mM, in a range of from 50 mM to 100 mM, or in a range of from 50 mM to 75 mM. If the formulation is to be subjected to lyophilization, then lower levels of NaCl from those provided above are preferred. The amount of rFVIII present in the formulations of Embodiment 4 may be the same as the amount provided in Embodiment 1.

The compositions also include a buffering agent such as histidine or MOPS buffer in a range of from 10 mM to 100 mM, in a range of from 10 mM to 60 mM, in a range of from 10 mM to 50 mM, in a range of from 10 mM to 40 mM, in a range of from 10 mM to 30 mM, in a range of from 12 mM to 30 mM, in a range of from 14 mM to 30 mM, in a range of from 16 mM to 30 mM, in a range of from 18 mM to 30 mM, in a range of from 12 mM to 28 mM, in a range of from 12 mM to 26 mM, in a range of from 12 mM to 24 mM, in a range of from 16 mM to 24 mM, in a range of from 18 mM to 24 mM, in a range of from 20 mM to 24 mM, or in a range of from 18 mM to 22 mM. The compositions also include a divalent cation such as calcium chloride in a range of from 1 mM to 20 mM, in a range of from 5 mM to 10 mM, in a range of from 1 mM to 30 mM, in a range of from 6 mM to 30 mM, in a range of from 7 mM to 30 mM, in a range of from 8 mM to 30 mM, in a range of from 5 mM to 20 mM, in a range of from 5 mM to 25 mM, or in a range of from 9 mM to 12 mM.

The compositions may also include a sugar or sugar alcohol such as sucrose or trehalose in a range of from 0.5% to 10%, in a range of from 0.6% to 10%, in a range of from 0.7% to 10%, in a range of from 0.8% to 10%, in a range of from 0.9% to 10%, in a range of from 1.0% to 10%, in a range of from 0.5% to 5%, in a range of from 0.6% to 5%, in a range of from 0.7% to 5%, in a range of from 0.8% to 5%, in a range of from 0.9% to 5%, in a range of from 1.0% to 5%, in a range of from 0.5% to 2.5%, in a range of from 0.6% to 2.5%, in a range of from 0.5% to 2.0%, in a range of from 0.5% to 1.5%, in a range of from 0.6% to 1.2%, in a range of from 0.8% to 1.2%, in a range of from 0.9% to 1.2%, or in a range of from 0.9% to 1.1%. The compositions may also include a non-ionic surfactant such as polysorbate 80 in a range of from 20 ppm to 250 ppm, in a range of from 50 ppm to 250 ppm, in a range of from 50 ppm to 150 ppm, in a range of from 60 ppm to 150 ppm, in a range of from 70 ppm to 150 ppm, in a range of from 80 ppm to 150 ppm, in a range of from 60 ppm to 140 ppm, in a range of from 60 ppm to 130 ppm, in a range of from 60 ppm to 120 ppm, in a range of from 60 ppm to 110 ppm, in a range of from 60 ppm to 100 ppm, in a range of from 70 ppm to 110 ppm, in a range of from 70 ppm to 105 ppm, in a range of from 70 ppm to 100 ppm, in a range of from 80 ppm to 100 ppm, or in a range of from 90 ppm to 110 ppm.

In certain versions of Embodiment 4 the FVIII formulation is free of histidine and/or HEPES and/or albumin, or contains less than 0.1%, less than 0.5%, less than 0.8%, less than 1.0%, or less than 5.0% by weight of histidine, and/or HEPES, and/or albumin. One version of Embodiment 3 is a FVIII formulation essentially free of histidine, HEPES and albumin.

### Embodiment 5

The invention also includes rFVIII formulations suitable for lyophilization. In certain versions of this embodiment, the FVIII formulations are particularly suitable for lyophilization of conjugated FVIII, PEGylated FVIII, PEGylated BDD, or PEGylated BDD mutants. The rFVIII formulations of this embodiment comprise (1) sodium chloride, and/or sucrose, and/or trehalose, (2) glycine and/or sucrose and/or trehalose; and (3) a divalent cation such as calcium chloride, and optionally contain (1) a nonionic surfactant, and/or (2) histidine, and if NaCl is present, then optionally also a sugar or a sugar alcohol, including without limitation sucrose and/or trehalose.

The invention includes formulations of Embodiment 5 as follows. A rFVIII formulation comprising:
(a) about 0 mM, or a range of from about 1 mM to about 20 mM, from about 1 mM to about 50 mM, from about 10 mM to about 50 mM, from about 10 mM to about 20 mM, from about 10 mM to about 30 mM, or from about 20 mM to about 50 mM histidine;
(b) a range of from 0.5% to 20%, a range of from 1.0% to 20%, a range of from 0.6% to 10%, a range of from 0.7% to 10%, a range of from 0.8% to 10%, a range of from 0.9% to 10%, a range of from 1.0% to 10%, a range of from 0.5% to 5%, a range of from 0.6% to 5%, a range of from 0.7% to 5%, a range of from 0.8% to 5%, a range of from 0.9% to 5%, a range of from 1.0% to 5%, a range of from 0.5% to 2.5%, a range of from 0.6% to 2.5%, a range of from 0.5% to 2.0%, a range of from 0.5% to 1.5%, a range of from 0.6% to 1.4%, a range of from 0.8% to 1.4%, a range of from 0.9% to 1.2%, a range of from 3.0% to 9.0%, a range of from 5.0% to 9.0%, a range of from 6.0% to 8.0%, a range of from 7.0% to 9.0%, or a range of from 0.9% to 1.1%, or about 1.0%, 1.1%, 1.2%, 1.3%, 1.4%, 1.5%, 1.6%, 1.7%, 1.8%, 1.9%, 2.0%, 3.0%, 4.0%, 5.0%, 6.0%, 7.0%, 7.5%, 8.0%, 8.5%, 9.0%, 9.5%, 10.0%, 12.0%, or 15.0% of sucrose or trehalose;
(c) a range of from about 1 mM to about 5 mM, from about 1 mM to about 3 mM, from about 1.5 mM to about 3.5 mM, or from about 1 mM to about 2.5 mM divalent cation such as a divalent calcium salt, including calcium chloride;
(d) about 0 mM, or a range of from about 10 mM to about 50 mM, from about 10 mM to about 40 mM, from about 10 mM to about 35 mM, from about 10 mM to about 30 mM; from about 10 mM to about 20 mM, from about 20 mM to about 50 mM, from about 20 mM to about 40 mM, or from about 20 mM to about 80 mM sodium chloride;
(e) about 0 mM, or a range of from about 20 ppm to about 50 ppm, from about 20 ppm to about 80 ppm, from about 50 ppm to about 80 ppm, from about 80 ppm to about 100 ppm, from about 80 ppm to about 200 ppm, or from about 50 ppm to about 100 ppm of a non-ionic surfactant, or about 60, 65, 70, 75, 80, 85, 90, 95, 100, 105, 110, 115, 120, 125, 130, 135, 140, 145, 150, 160, 170, 180, 190, or 200 ppm of a non-ionic surfactant;
(f) about 0%, or a range of from about 1.0% to about 5.0%, a range of from about 1.0% to about 4.0%, a range of from about 1.0% to about 3.0%, a range of from about 1.0% to about 2.0%, a range of from about 1.0% to about 1.5%, a range of from about 1.0% to about 1.4%, a range of from about 0.5% to about 5.0%, a range of from about 0.5% to about 4.0%, a range of from about 0.5% to about 3.0%, a range of from about 0.5% to about 2.0%, a range of from about 0.5% to about 1.5% glycine, or about 1.5%, 1.8%, 2.0%, 2.1%, 2.2%, 2.3%, 2.4%, 2.5%, 2.6%, 2.7%, 2.8%, 2.9%, 3.0%, 3.3%, 3.5%, or 4.0% glycine and
(g) a range of from about 100 IU/ml to about 5000 IU/ml, from about 100 IU/ml to about 2000 IU/ml, from about 100 IU/ml to about 3000 IU/ml, from about 100 IU/ml to about 4000 IU/ml, from about 100 IU/ml to about 1200 IU/ml, from about 250 IU/ml to about 5000 IU/ml, from about 250 IU/ml to about 1000 IU/ml, from about 250 IU/ml to about 2000 IU/ml, from about 250 IU/ml to about 3000 IU/ml, from about 500 IU/ml to about 1000 IU/ml, from about 500 IU/ml to about 3000 IU/ml, from about 1000 IU/ml to about 2000 IU/ml, from about 1000 IU/ml to about 3000 IU/ml, from about 1000 IU/ml to about 4000 IU/ml, or from about 1000 IU/ml to about 5000 IU/ml, or about 100, 200, 300, 400, 500, 600, 700, 800, 900, 1000, 1200, 1400, 1600, 1800, 2000, 2200, 2400, 2600, 2800, 3000, 3200, 3500, 3800, 4000, 4200, 4500, 4600, 4800, 5000, 5500, or 6000 IU/ml of rFVIII; wherein the rFVIII formulation has a pH in a range of from about pH 6.0 to about pH 6.5, from about pH 6.0 to about pH 7.0, from about pH 6.0 to about pH 7.5, from about pH 6.5 to about pH 7.5, or from about pH 7.0 to about pH 7.5, or a pH of about pH 6.0, 6.5, 7.0, 7.1, 7.2, 7.3, 7.4 or about pH 7.5.

In one version of Embodiment 5, the rFVIII formulation comprises sodium chloride and contains less than 2.0% sucrose or sucrose in a range of from 0.5% to 2.0%, and contains less than 1.0%, less than 0.5%, less than 0.1% or no trehalose. In this version, NaCl may be present at a range of from about 10 mM to about 50 mM, from about 10 mM to about 40 mM, from about 10 mM to about 35 mM, from about 10 mM to about 30 mM; from about 10 mM to about 20 mM, from about 20 mM to about 50 mM, from about 20 mM to about 40 mM, or from about 20 mM to about 80 mM sodium chloride. In this version of Embodiment 5, glycine is present at a range of from about 1.0% to about 5.0%, a range of from about 1.0% to about 4.0%, a range of from about 1.0% to about 3.0%, a range of from about 1.0% to about 2.0%, a range of from about 1.0% to about 1.5%, a range of from about 1.0% to about 1.4%, a range of from about 0.5% to about 5.0%, a range of from about 0.5% to about 4.0%, a range of from about 0.5% to about 3.0%, a range of from about 0.5% to about 2.0%, a range of from about 0.5% to about 1.5%, or at about 1.5%, 1.8%, 2.0%, 2.1%, 2.2%, 2.3%, 2.4%, 2.5%, 2.6%, 2.7%, 2.8%, 2.9%, 3.0%, 3.3%, 3.5%, or 4.0% and sucrose is present at a range of from 0.5% to 5%, a range of from 0.6% to 5%, a range of from 0.7% to 5%, a range of from 0.8% to 5%, a range of from 0.9% to 5%, a range of from 1.0% to 5%, a range of from 0.5% to 2.5%, a range of from 0.6% to 2.5%, a range of from 0.5% to 2.0%, a range of from 0.5% to 1.5%, a range of from 0.6% to 1.4%, a range of from 0.8% to 1.4%, a range of from 0.9% to 1.2%, or a range of from 0.9% to 1.1%, or about 1.0%, 1.1%, 1.2%, 1.3%, 1.4%, 1.5%, 1.6%, 1.7%, 1.8%, 1.9%, 2.0%, 3.0%, 4.0% sucrose. In this version of Embodiment 5, histidine is present at a range of from about 1 mM to about 20 mM, from about 1 mM to about 50 mM, from about 10 mM to about 50 mM, from about 10 mM to about 20 mM, from about 10 mM to about 30 mM, or from about 20 mM to about 50 mM and a non-ionic surfactant such as polysorbate 20 or polysorbate 80 is present at a range of from about 20 ppm to about 50 ppm, from about 20 ppm to about 80 ppm, from about 50 ppm to about 80 ppm, from about 80 ppm to about 100 ppm, from about 80 ppm to about 200 ppm, or from about 50 ppm to about 100 ppm of a non-ionic surfactant, or about 60, 65, 70, 75, 80, 85, 90, 95, 100, 105, 110, 115, 120, 125, 130, 135, 140, 145, 150, 160, 170, 180, 190, or 200 ppm. In this version of Embodiment 5, trehalose is present at less than 1.0%, less than 0.5%, less than 0.1% by weight or is not present.

In another version of Embodiment 5, sodium chloride is present at less than 1.0%, less than 0.5%, less than 0.1% by weight or is not present. In this version, sucrose or trehalose is present a range of from 0.5% to 20%, a range of from 1.0% to 20%, a range of from 0.6% to 10%, a range of from 0.7% to 10%, a range of from 0.8% to 10%, a range of from 0.9% to 10%, a range of from 1.0% to 10%, a range of from 3.0% to 9.0%, %, a range of from 5.0% to 9.0%, a range of from 6.0% to 8.0%, %, or a range of from 7.0% to 9.0%, or about 5.0%, 6.0%, 7.0%, 7.5%, 8.0%, 8.5%, 9.0%, 9.5%, 10.0%, or 12.0%. In this version of Embodiment 5, glycine is present at less than 1.0%, less than 0.5%, less than 0.1% by weight or is not present.

Aspects of the present disclosure may be further understood in light of the following examples, which should not be construed as limiting the scope of the present teachings in any way.

### EXAMPLES

### Example 1

### Effect of Sodium Chloride, Polysorbate 80, and Human Serum Albumin on BDD-rFVIII Protein Solubility and Stability

### Effect of Sodium Chloride

Studies were performed on BDD mutants having introduced cysteine residues that permit the stabilization of FVIII by formation of at least one disulfide bond between different domains of FVIII. In particular, BDD-SQ (SEQ ID NO: 3) was mutated at Tyr664Cys:Thr1826Cys to create the C664-BDD mutant used in this example. For methods of preparation, see U.S. Pat. No. 7,928,199 (Griffin et al.). When the C664-BDD mutant was formulated in a buffer containing histidine, unacceptable levels of precipitation were observed.

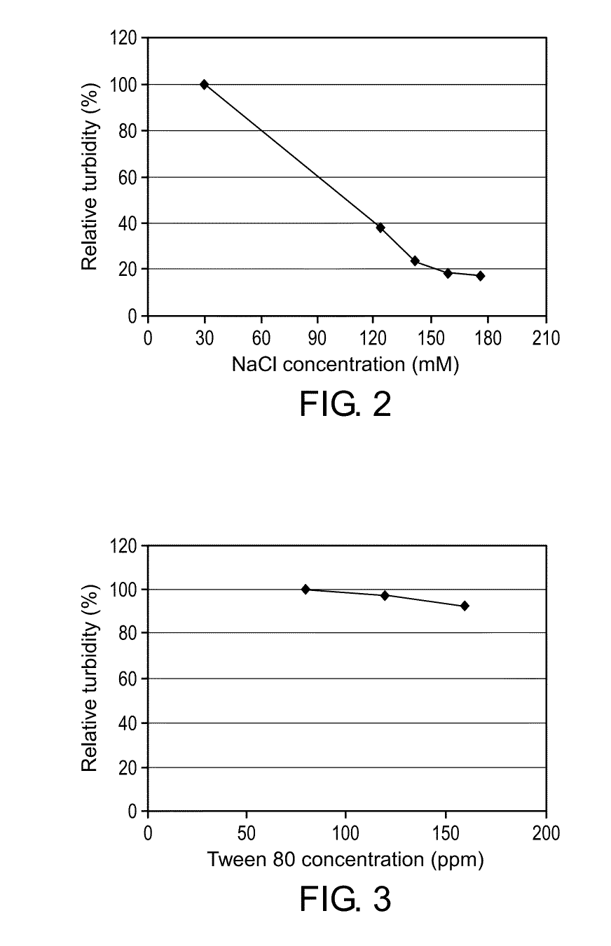

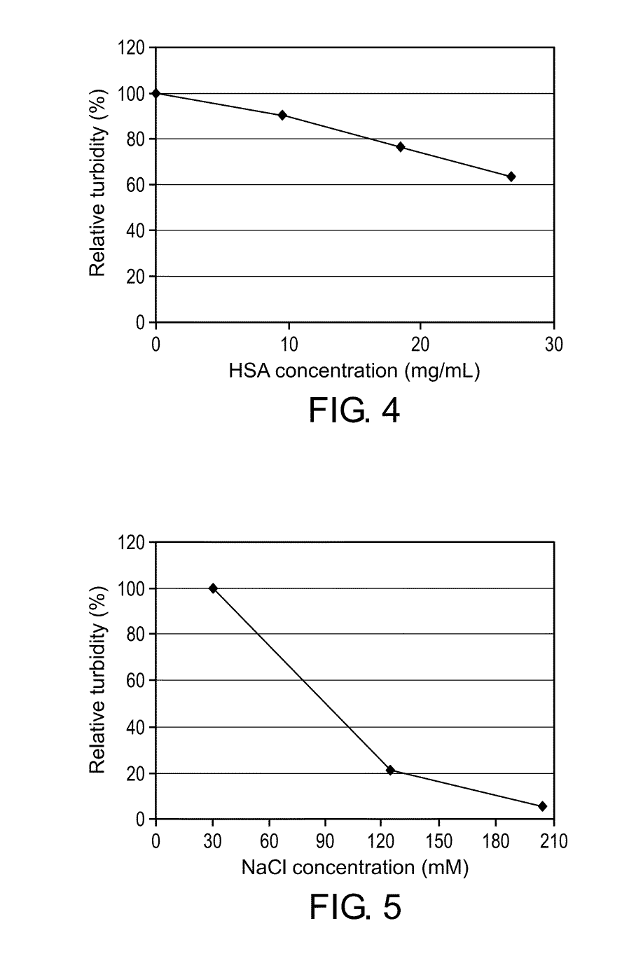

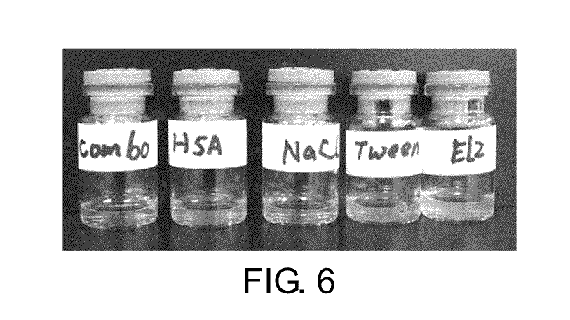

A study was performed to determine whether the precipitation observed when the C664-BDD mutant was placed in histidine buffer could be reversed. The buffer solution in which precipitation was observed contained 20 mM histidine, 30 mM sodium chloride, 2.5 mM calcium chloride, 29 mM sucrose, 293 mM glycine and 80 ppm polysorbate 80. The C664 BDD mutant was present at 145 IU/ml. The aim of the study was to develop a formulation that stabilizes BDD-rFVIII mutants. Solubilizers and stabilizers, such as sodium chloride, Polysorbate 80, and human serum albumin (HSA) were tested to either increase the solubility of the mutants or to improve the stability by reducing protein aggregation. Results are shown in FIGS. 2-6. The experiments shown in FIGS. 2 and 5 both involved modification of the NaCl concentration, and the results in each instance showed remarkable turbidity decline from a solution containing 30 mM NaCl when compared to a solution containing about 120 mM NaCl. The study established that as the sodium chloride concentration increased, the turbidity of the solution comprising the mutants decreased, suggesting that sodium chloride reversed the precipitation process. When the sodium chloride concentration was 176 mM or higher, the cloudy solution turned to a clear solution and the turbidity dropped from 0.169 to 0.029, which is more than 80% based on A_{340 nm} measurements (FIG. 2). These results demonstrated that sodium chloride was an effective solubilizer for the BDD-rFVIII mutants and can reverse their precipitation. In summary, higher sodium chloride concentrations improved the solubility of the BDD-rFVIII mutants. Table 2 shows preferred formulations. “BDD-rFVIII mutants” in Table 2 refers to a formulation of BDD-SQ mutated at Tyr664Cys:Thr1826Cys. “Full-length rFVIII” in Table 2 refers to a formulation of FVIII that has the amino acid sequence of SEQ ID NO: 2 (full-length FVIII).

TABLE 2
Formulation Composition for full-length rFVIII and
BDD-rFVIII mutants

| Composition | BDD-rFVIII mutants | Full-length rFVIII |
|---|---|---|
| Sodium chloride (mM) | 220 | 30 |
| Sucrose (mM) | 29 | 29 |
| Histidine (mM) | 20 | 20 |
| Glycine (mM) | 293 | 293 |
| Calcium chloride (mM) | 2.5 | 2.5 |
| Polysorbate 80 (ppm) | 80 | 80 |

### Example 2

### Formulation Development for rFVIII PEGylation Through Random Lysine Coupling

PEG polymer was conjugated to the full-length rFVIII of SEQ ID NO: 1 using random lysine coupling. In this type of coupling, the reactive groups are primarily the N-terminal amine or the ε-amino group of lysine in a protein. Other primary or secondary amine groups in the formulation could interfere with the reaction. Because many full-length and BDD-rFVIII formulations comprise amino acids, such as glycine and histidine, new formulations were developed for PEGylation of these molecules. While glycine was used as a bulking agent in the full-length rFVIII formulation and could be eliminated during PEGylation, histidine served as a buffer component and needed to be replaced with another buffer.

A suitable buffer system meets the following criteria: (1) it provides buffer capacity at pH 6-7; (2) it does not form insoluble complex or chelate with calcium chloride, an important rFVIII stabilizer; and (3) it does not comprise primary or secondary amine groups.

Several commonly used buffers were considered for random PEGylation of rFVIII. As shown in Table 3, only two buffer systems, tri-ethanolamine (“TEA”) and MOPS were selected for further investigation.

TABLE 3
Buffers Considered for Random PEGylation of rFVIII

| | | | | pH change |
|---|---|---|---|---|
| | | Ca²⁺ | | during |
| Buffer at pH 7 | Ca²⁺ ppt. | chelating | Amine group | freezing |
| Citrate | | X | | |
| Phosphate | X | | | X |
| Histidine | | | X | |
| TRIS | | | X | |
| Carbonate | X | | | |
| Triethanolamine | | | | |
| (TEA) | | | | |
| MOPS or | | | | |
| MOPSO | | | | |
| HEPES | | | X | |

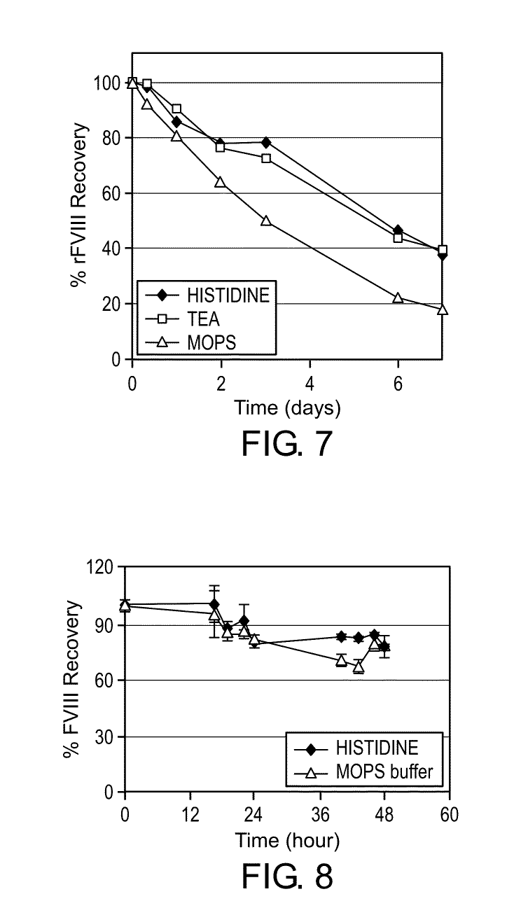

For this study, full-length rFVIII was dialyzed against the formulations listed in Table 4. The dialyzed rFVIII in the three formulations was placed at 40° C. (FIG. 7) or 25° C. (FIG. 8) to establish stability at accelerated conditions and the results are shown in FIGS. 7 and 8.

TABLE 4
Buffers Evaluated for Random PEGylation of rFVIII

| | | | Tween | | | Sodium | Buffer |
|---|---|---|---|---|---|---|---|
| | NaCl | CaCl₂ | 80 | Glycine | Sucrose | Azide | Agent |
| | (mM) | (mM) | (ppm) | (mM) | (mM) | (%) | (20 mM) |
| 1 | 30 | 2.5 | 80 | — | 29 | 0.05 | TEA |
| 2 | 30 | 2.5 | 80 | — | 29 | 0.05 | MOPS |
| 3 | 30 | 2.5 | 80 | 293 | 29 | 0.05 | Histidine |

### Example 3

### PEGylation for BDD-rFVIII

BDD-rFVIII encounters formulation challenges due to its propensity for aggregation. Therefore, one of the objectives with designing a formulation for PEGylated rFVIII was to ensure its stability in solution. The working formulation for the PEGylated BDD-rFVIII comprised 200 mM sodium chloride, 20 mM MOPS, 10 mM CaCl₂, 100 ppm polysorbate 80 and 29 mM sucrose. 200 mM sodium chloride will impose difficulties during freeze-drying. Accordingly, the solubility and potency of the PEGylated BDD-rFVIII were evaluated as a function of sodium chloride concentration in the range of 50 and 250 mM.

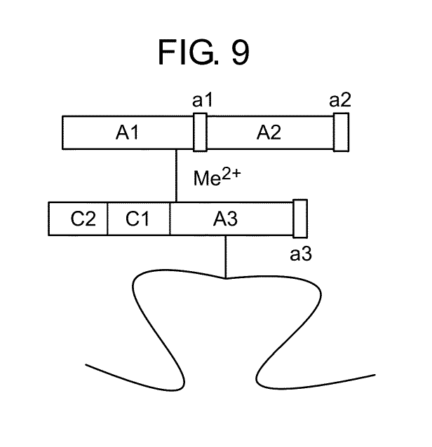

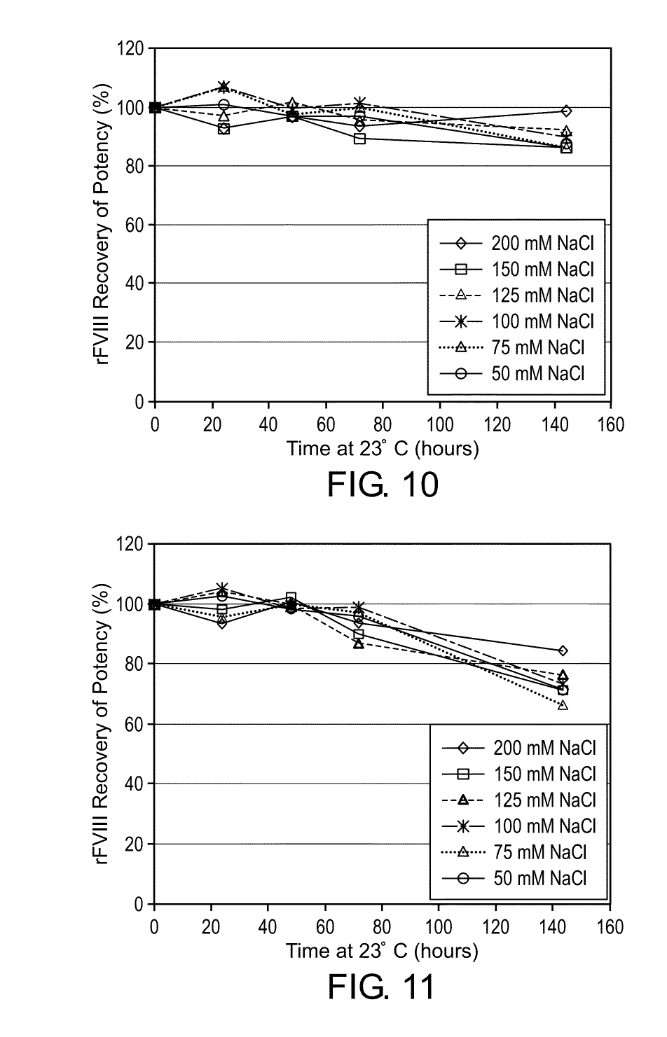

The buffer composition used for the study is shown in Table 5 and the data are summarized in FIGS. 10 and 11. The PEGylated BDD used in this example comprised the amino acid sequence of SEQ ID NO: 3 with one amino acid mutation to create a free cysteine at which PEG was added. This is shown graphically in FIG. 9. The PEGylated BDD-rFVIII retained more than 87% potency in the formulation comprising 50-150 mM sodium chloride during 6 days storage at 23° C. UnPEGylated BDD-rFVIII retained 70% potency in the same formulation during 6 days storage at 23° C. Both molecules remained soluble during the study with no visual detection of precipitates or opalescence. These and earlier data suggest that 100 mM sodium chloride can be used for further formulation development.

TABLE 5
Composition of the Formulation Used for Evaluating the
Effect of Sodium Chloride

| | | | Polysorbate | |
|---|---|---|---|---|
| MOPS | NaCl | CaCl2 | 80 | Sucrose |
| (mM) | (mM) | (mM) | (ppm) | (mM) |
| 20 | 250 | 10 | 100 | 29 |
| 20 | 200 | 10 | 100 | 29 |
| 20 | 150 | 10 | 100 | 29 |
| 20 | 100 | 10 | 100 | 29 |
| 20 | 50 | 10 | 100 | 29 |
| 20 | 25 | 10 | 100 | 29 |
| 20 | 0 | 10 | 100 | 29 |

The effect of sodium chloride on the solubility and aggregation of PEGylated and unPEGylated BDD-rFVIII was investigated.

UV absorbance of PEGylated BDD-rFVIII in MOPS buffer comprising 25 mM, 55 mM, 75 mM, 125 mM and 200 mM sodium chloride showed no scattering of the PEGylated BDD-rFVIII at all sodium chloride concentration tested, suggesting lack of aggregation. In contrast, the unPEGylated-rFVIII showed considerable scattering at 25 mM, 55 mM and 75 mM sodium chloride most likely due to formation of soluble aggregates. When sodium chloride concentration was increased to 125 mM and 200 mM, no scattering was observed. It was concluded, therefore, that higher salt concentrations prevented aggregate formation.

### Example 4

### Development of Freeze-Drying Formulation for PEGylated BDD-rFVIII

Four candidate formulations were screened for lyophilization of PEGylated BDD-rFVIII. The PEGylated BDD used in this example comprised the amino acid sequence of SEQ ID NO: 3 with one amino acid mutation to create a free cysteine at which PEG was added. The aim was to evaluate the stability of the lyophilized drug product in these formulations and to select a formulation for the leading stability study. The formulations that were screened were (1) Formulation A, which had been successful for unPEGylated full-length rFVIII, (2) Formulation B, comprising increased solids content compared to Formulation A, (3) Formulation C with sucrose instead of the NaCl used in Formulation A, and (4) Formulation D with trehalose instead of the NaCl used in Formulation A. The last two formulations provided an amorphous matrix for the lyophilized drug product.

Stability was evaluated at three storage temperatures (5° C., 25° C. and 40° C.). Table 6 shows the formulation composition for PEGylated BDD-rFVIII used for stability evaluation.

The concentrations of sucrose and glycine were increased from 29 mM and 293 mM in Formulation A to 38 mM and 346 mM in Formulation B. The additional solids were added to enhance the mechanical strength of the freeze-dried cake and improve the appearance of the final drug product.

TABLE 6
Formulation Composition for PEGylated BDD-rFVIII Used in Stability Evaluation

| Component | Formulation A | | Formulation B | | Formulation C | | Formulation D | |
|---|---|---|---|---|---|---|---|---|
| Calcium | 2.5 | mM | 2.5 | mM | 2.5 | mM | 2.5 | mM |
| Chloride | | | | | | | | |
| Sodium | 30 | mM | 30 | mM | X | | X | |
| Chloride | | | | | | | | |
| Histidine | 20 | mM | 20 | mM | 20 | mM | 20 | mM |
| Glycine | 293 | mM | 346 | mM | X | | X | |
| Polysorbate 80 | 80 | ppm | 80 | ppm | 80 | ppm | 80 | ppm |
| Sucrose | 29 | mM | 38 | mM | 234 | mM | X | |
| Trehalose | X | | X | | X | | 211 | mM |
| PEGylated | 100 | IU/mL | 100 | IU/mL | 100 | IU/mL | 100 | IU/mL |
| BDD-rFVIII | | | | | | | | |
| concentration | | | | | | | | |
| (IU/mL) | | | | | | | | |

1: pH = 6.8 for all formulations

Formulations C and D were designed to provide an alternate matrix compared to the other two formulations. Formulations A and B formed a crystalline matrix upon freeze-drying due to the presence of sodium chloride and glycine as structural stability and bulking agents. The concentrations of sucrose and trehalose were increased to 234 mM and 211 mM, respectively, in lieu of including sodium chloride and glycine. This resulted in an amorphous matrix for the freeze-dried drug product.

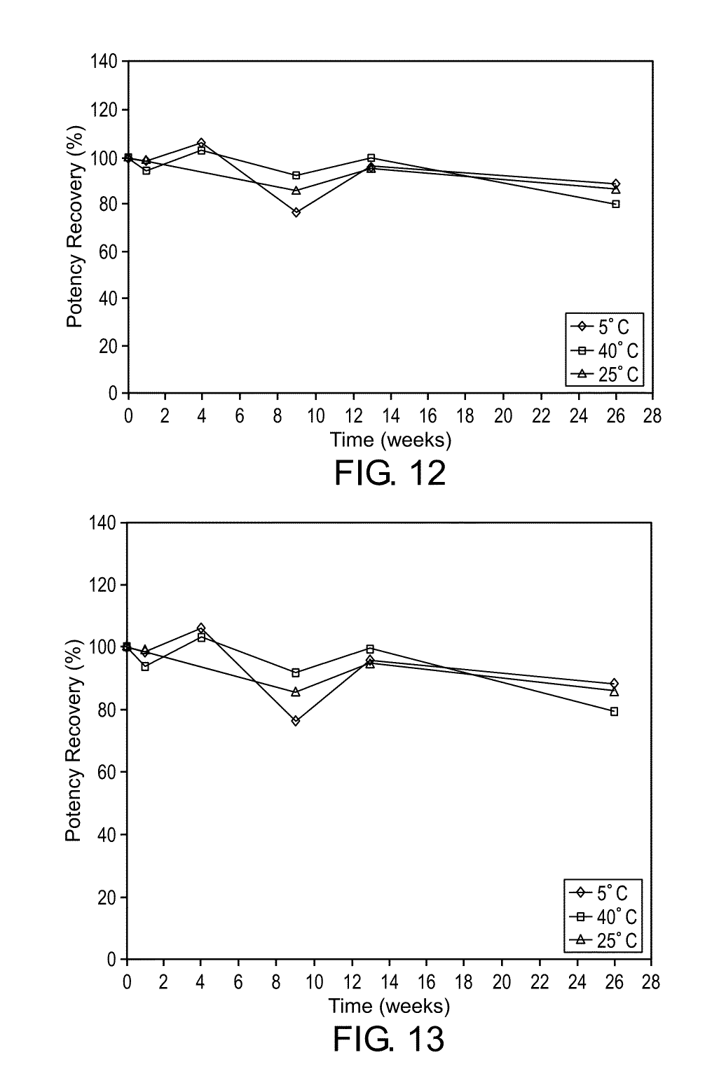

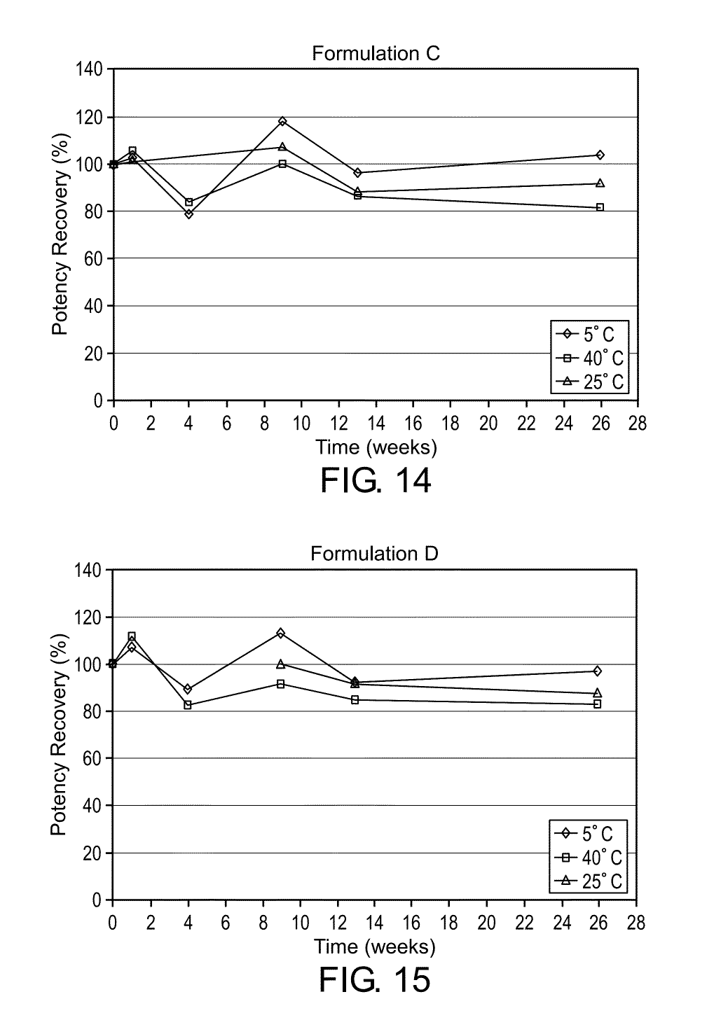

The stability program for each of the four candidate formulations was set up for a 26 week time period. Stability was evaluated by potency, moisture content, percent high molecular weight (HMW) impurities and total product related impurities by SEC-HPLC. The potency recovery data for the four formulations are summarized in FIGS. 12-15.

The data of potency recovery, moisture content by Karl Fischer, and percent aggregates and product related impurities by SEC-HPLC (tested at 26 weeks) for the four formulations demonstrate that rFVIII is stable in the four formulations.

Stability for PEGylated BDD-rFVIII was further evaluated with Formulations A and B (see Table 6 for formulation composition). Two drug product lots were prepared at lab-scale and were placed on stability at 5° C. and 25° C. and 40° C. Potency by the chromogenic assay, percent high molecular weight impurities and total product related impurities by SEC-HPLC, and moisture by Karl Fischer were employed for drug product stability evaluation. Target concentrations and ranges of the components used in Formulation A are presented in Table 7.

TABLE 7
Target Concentrations and Ranges of the Components Used in the
Formulation A

| | Formulation A | | Low and High Concentration |
|---|---|---|---|
| Component | Target Concentrations | | Range |
| Calcium | 2.5 | mM | 1.5 mM to 3.5 mM |
| Chloride | | | |
| Sodium | 30 | mM | 21 mM to 43 mM |
| Chloride | | | |
| Histidine | 20 | mM | 15 mM to 27 mM |
| Glycine | 293 | mM | 240 mM to 386 mμM |
| Polysorbate 80 | 80 | ppm | 57 ppm to 103 ppm |
| Sucrose | 29 | mM | 20 mM to 41 mM |
| PEGylated | 200 | IU/mL | 188 IU/mL to 250 IU/mL |
| BDD-rFVIII | 400 | IU/mL | 376 IU/mL to 500 IU/mL |
| concentration | 1200 | IU/mL | 1128 IU/mL to 1500 IU/mL |
| (IU/mL) | | | |

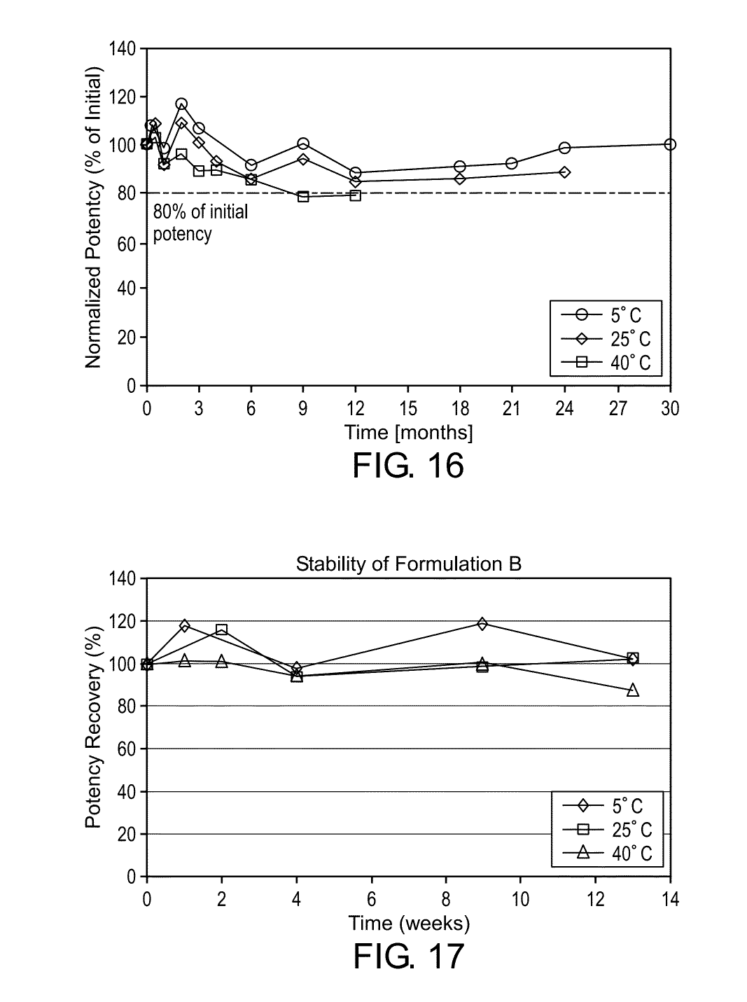

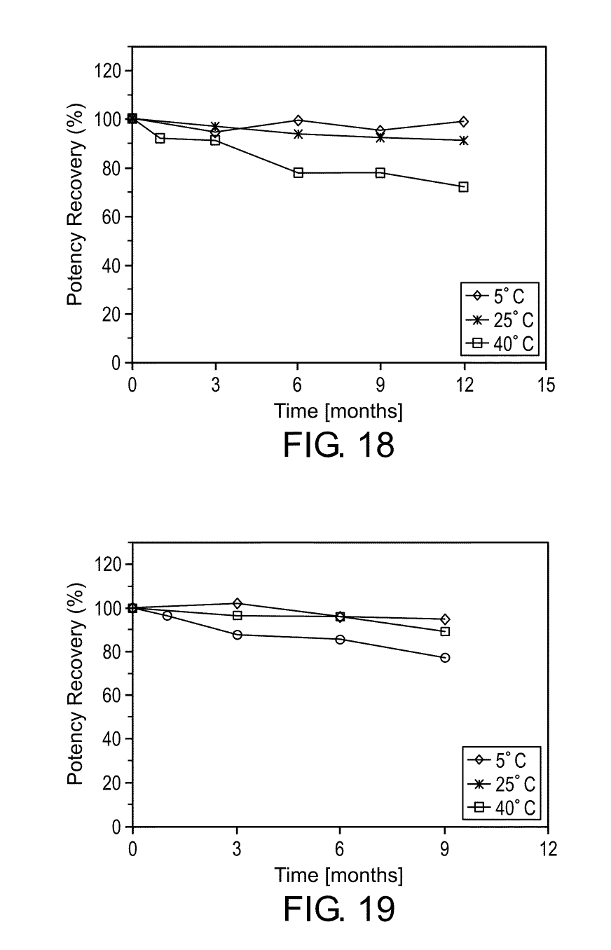

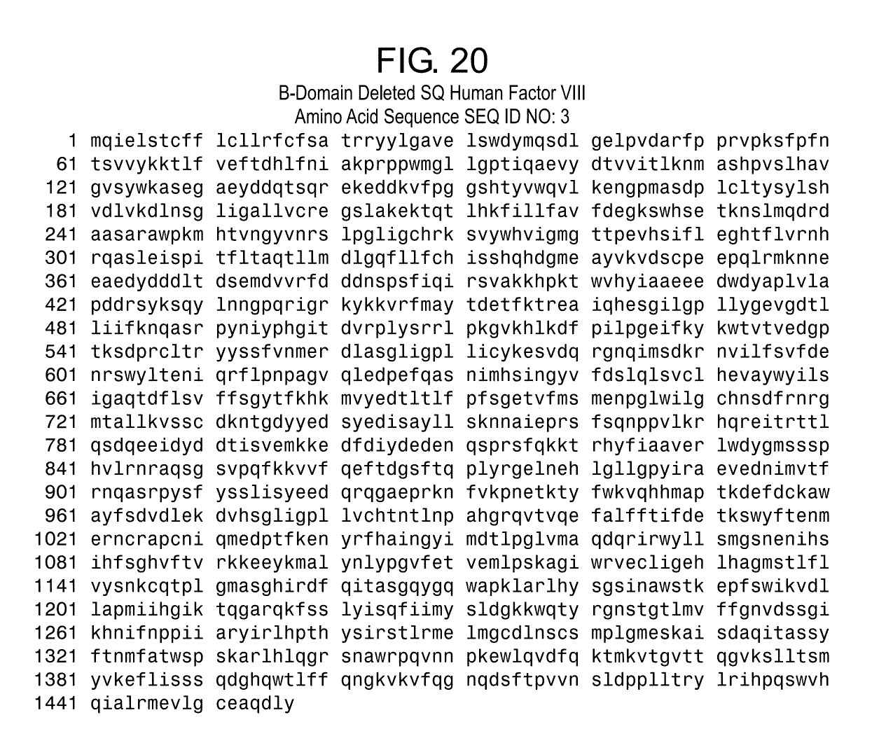

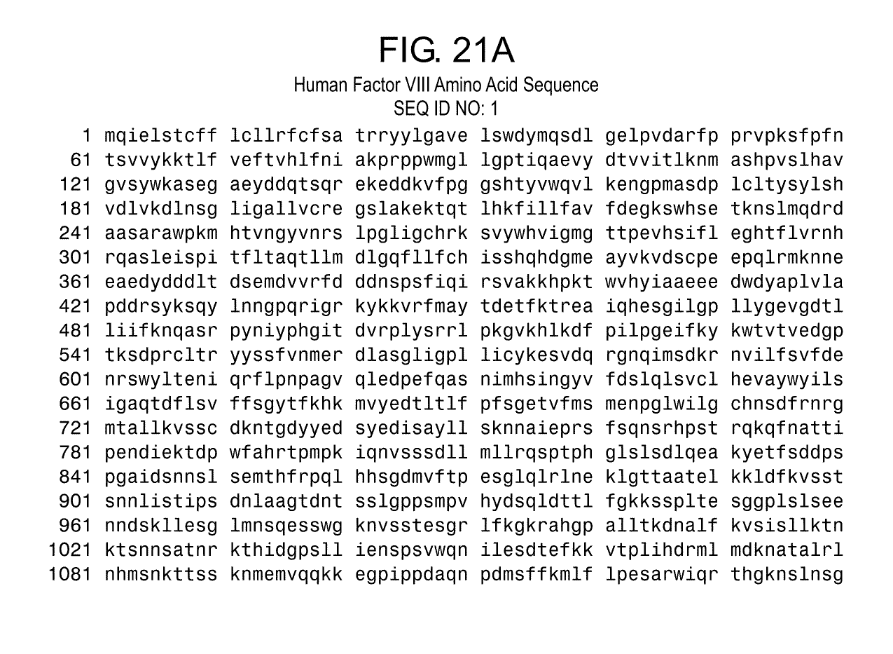

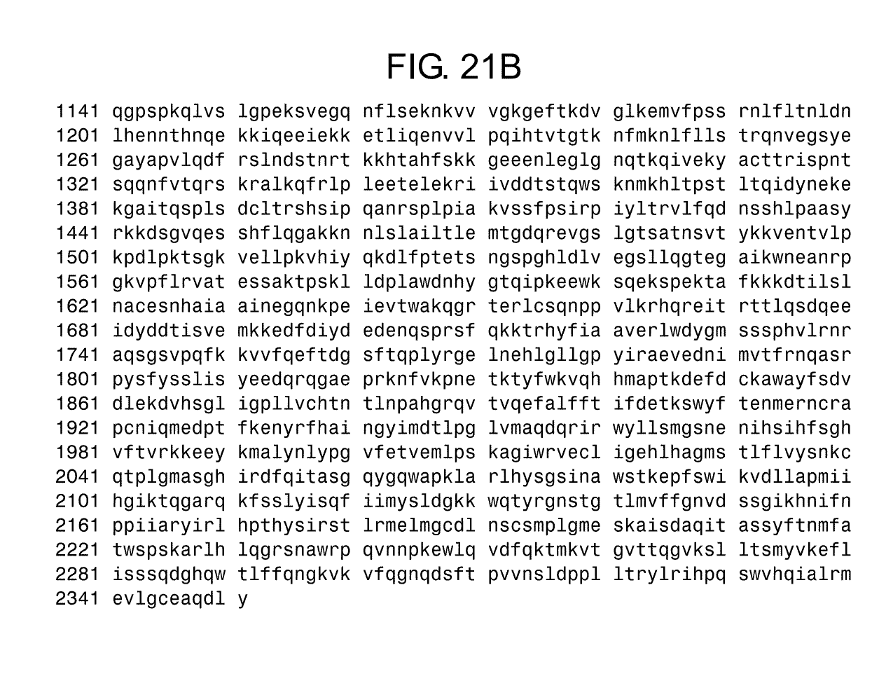

These data demonstrated comparable drug product stability in the two formulations. The study with Formulation A was continued up to 30 months, whereas the study with Formulation B was terminated at 3 months (FIGS. 16 and 17, respectively). rFVIII concentration in FIGS. 18 and 19 was 400 IU/mL.

Formulation A was selected for further development and was tested with PEGylated rFVIII at concentrations of 200 IU IU/mL and 1200 IU/mL. The potency profiles at 200 IU/mL and 1200 IU/mL are shown in FIGS. 18 and 19, respectively. The data demonstrate that Formulation A provides continuous stability for the PEGylated rFVIII.

1. A rFVIII formulation comprising:
(a) a range of from about 1 mM to about 5 mM divalent cation;
(b) a range of from about 150 mM to about 250 mM sodium chloride or potassium chloride;
(c) a range of from about 50 ppm to about 200 ppm of a non-ionic surfactant; and
(d) a range of from about 100 IU/ml to about 5000 IU/ml of a rFVIII, wherein the rFVIII comprises an amino acid sequence that has one or more non-cysteine residues in the amino acid sequence of SEQ ID NO: 3 replaced with cysteine residues such that at least one pair of cysteine residues creates a disulfide bond not found in wild type FVIII;
wherein the rFVIII formulation has a pH in a range of from about pH 6.0 to about pH 7.5.

2. The rFVIII formulation of claim 1 further comprising:
(a) a range of from about 10 mM to about 50 mM histidine;
(b) a range of from about 10 mM to about 100 mM of a sugar or sugar alcohol; and
(c) a range of from about 150 mM to about 400 mM glycine.

3. A rFVIII formulation comprising:
(a) a range of from 10 mM to 100 mM MOPS;
(b) a range of from 0.5% to 10% by weight of a sugar or a sugar alcohol;
(c) a range of from 0.5 mM to 20 mM of a divalent cation;
(d) a range of from 10 mM to 100 mM sodium chloride or potassium chloride;
(e) a range of from 50 to 150 ppm of a non-ionic surfactant; and
(f) a range of from about 1000 IU/ml to about 1500 IU/ml of rFVIII;
wherein the rFVIII formulation contains less than 5.0% by weight of components other than rFVIII having primary or secondary amine groups.

4. The rFVIII formulation of claim 3 that is essentially free of histidine and glycine.

5. A method for the covalent conjugation of rFVIII to a biocompatible polymer comprising:
(a) obtaining the rFVIII formulation of claim 3;
(b) adding a functionalized polymer to create a reaction mixture, wherein the polymer is functionalized with a chemical moiety reactive with amine groups on rFVIII; and
(c) incubating the reaction mixture under conditions of time and temperature such that covalent attachment of the polymer to the rFVIII occurs.

6. A rFVIII formulation comprising:
(a) a range of from 10 mM to 100 mM MOPS;
(b) a range of from 150 mM to 300 mM NaCl;
(c) a range of from 1 mM to 20 mM divalent cation; and
(d) a range of from about 100 IU/ml to about 5000 IU/ml of nonconjugated rFVIII.

7. The rFVIII formulation of claim 6 further comprising
(a) a range of from 0.5% to 10% of a sugar or sugar alcohol; and
(b) a range of from 20 ppm to 250 ppm of a non-ionic surfactant.

8. A rFVIII formulation comprising:
(a) a range of from 10 mM to 100 mM MOPS or histidine;
(b) a range of from 25 mM to 200 mM NaCl;
(c) a range of from 1 mM to 20 mM divalent cation; and
(d) a range of from about 100 IU/ml to about 5000 IU/ml of conjugated rFVIII.

9. The rFVIII formulation of claim 8 further comprising:
(a) a range of from 0.5% to 10% of a sugar or sugar alcohol; and
(b) a range of from 20 ppm to 250 ppm of a non-ionic surfactant.

10. A rFVIII formulation comprising:
(a) about 0 mM, or a range of from about 1 mM to about 20 mM histidine;
(b) a range of from 0.5% to 20% of sucrose or trehalose;
(c) a range of from about 1 mM to about 5 mM divalent cation;
(d) about 0 mM, or a range of from about 10 mM to about 50 mM sodium chloride;
(e) about 0 mM, or a range of from about 20 ppm to about 80 ppm of a non-ionic surfactant;
(f) about 0%, or a range of from about 1.0% to about 5.0%, glycine and
(g) a range of from about 100 IU/ml to about 5000 IU/ml of conjugated rFVIII;
wherein the rFVIII formulation has a pH in a range of from about pH 6.0 to about pH 7.5.

11. The rFVIII formulation of claim 10, wherein
(a) sodium chloride is present in a range of from about 10 mM to about 50 mM;
(b) sucrose is present in a range of a range of from 0.5% to 2.0%,
(c) glycine is present at a range of from about 1.0% to about 5.0%,
(d) histidine is present in a range of from about 1 mM to about 20 mM, and
(e) a non-ionic surfactant is present in a range of from about 20 ppm to about 80 ppm.

12. The rFVIII formulation of claim 10, wherein
(a) sodium chloride is present at less than 1.0% by weight or is not present;
(b) sucrose or trehalose is present in a range of from 0.5% to 20%, and
(c) glycine is present at less than 1.0% by weight or is not present.

13. The rFVIII formulation of claim 12, wherein sucrose or trehalose is present at a range of from 1.0% to 10.0%.

14. A method of treating hemophilia A comprising administering a therapeutically effective amount of a rFVIII formulation of claim 1 to a patient in need thereof.


SEQUENCE LISTING (5 sequences)

SEQ ID NO: 1 - protein, 2351 aa, Homo sapiens [sequence omitted]

SEQ ID NO: 2 - protein, 2332 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 1457 aa, Artificial Sequence
  Deletion variant of human FVIII
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 4 aa, Homo sapiens
SFSQ

SEQ ID NO: 5 - protein, 10 aa, Homo sapiens
NPPVLKRHQR